(19) United States
(12) Patent Application Publication
(10) Pub. No.: US 2024/0196931 A1
(43) Pub. Date: Jun. 20, 2024

(54) BOTANICAL SUPPLEMENT AND METHOD FOR PREVENTING DISEASES, PROMOTING GROWTH AND SURVIVAL OF AN ANIMAL BY USING THE SAME

(71) Applicant: ACADEMIA SINICA, Taipei (TW)

(72) Inventors: Lie-Fen SHYUR, Taipei (TW); Yu-Chuan LIANG, Taipei (TW); Wen-Yuh LIN, Taipei (TW); Chin-Ting CHANG, Taipei (TW); Meng-Ting CHANG, Taipei (TW); Yu-Hsin CHEN, Taipei (TW)

(21) Appl. No.: 18/286,943

(22) PCT Filed: Apr. 14, 2022
(86) PCT No.: PCT/US2022/024730

§ 371 Date: Oct. 13, 2023

(51) Int. Cl. A23K 10/30; A23K 50/30; A61K 36/28; A61P 31/14

(52) U.S. Cl. CPC A23K 10/30; A23K 50/30; A61K 36/28; A61P 31/14

(57) ABSTRACT

Provided is an animal feed composition including a plant-based feed additive. Also provided is a method for promoting overall health condition of an animal, including increase of feed efficiency and decrease of feed converting rate, increase of probiotics and decrease of pathogens in gut microbiota of pigs, anti-inflammation, affecting primary metabolism, promoting growth and survival rate, and preventing or treating diseases caused by pathogenic bacteria, porcine reproductive and respiratory syndrome virus (PRRSV).

### TECHNICAL FIELD

The present disclosure relates generally to Crassocephalum rabens or an extract thereof and methods for providing the same in preventing or treating diarrhea and increasing body weight gain and survival rate in weaned pigs, and more specifically to methods for providing Crassocephalum rabens or an extract thereof as a non-antibiotic growth promoter and phytogenics for weaned pigs.

### BACKGROUND

The weaning stage is the most challenging phase in domestic pig farming, often accompanied by reduced growth performance and increased incidences of diarrhea.

Weaning is usually conducted at around 3 to 4 weeks old in the modem swine industry, and piglets confront the transition from a liquid-based diet to a solid-based diet, a sudden, complex and highly stressful event in the life of a piglet. Weaning piglets are usually vulnerable to nutritional, physiological, and psychological stressors that lead to alterations in intestinal morphology, physiological function, and a shift in the intestinal microbiome (Thomas A. Marsteller and Brad Fenwick, “Actinobacillus pleuropneumoniae disease and serology,” Swine Health and Production. 1999 (7): 161-165; Zhuofei Xu, et al., “Comparative genomic characterization of Actinobacillus pleuropneumoniae,” Journal of Bacteriology 2010 (192): 5625-5636; Joy M Campbell, Joe D Crenshaw and Javier Polo, “The biological stress of early weaned piglets,” Journal of Animal Science and Biotechnology 2013(4): 19). The disruption of gut microbiota is regarded as one of the major factors resulting in post-weaning diarrhea (Raphaele Gresse et al., “Gut microbiota dysbiosis in postweaning piglets: understanding the keys to health,” Trends in Microbiology 2017 (25): 851-873).

Over the past several decades, antibiotics and other forms of anti-microbial agents (AMAs) have been recognized as antimicrobial growth promoters (AGPs) to promote growth performance throughout gestation, farrowing, weaning and finishing stages for prevention of diarrhea and dysentery (Christy Manyi-Loh et al., “Antibiotic use in agriculture and its consequential resistance in environmental sources: potential public health implications,” Molecules 2018 (23): 795). However, accumulated evidence also reveals concerns in relation to development of antibiotic resistant microbiota and their associated resistance genes that have unexpected detrimental effects on livestock and human health (Lauren Brinkac et al., “The threat of antimicrobial resistance on the human microbiome,” Microbial Ecology 2017 (74): 1001-1008; Zachary M. Burcham, et al., “Detection of critical antibiotic resistance genes through routine microbiome surveillance,” PloS One 2019 (14): e0213280; David A. Relman and Marc Lipsitch, “Microbiome as a tool and a target in the effort to address antimicrobial resistance,” Proceedings of the National Academy of Sciences 2018 (115): 12902-12910).

Accordingly, European Union (EU) has banned the use of AGPs in livestock and poultry production since Jan. 1, 2006, and the U.S. Food and Drug Administration (FDA) also banned the use of antibiotics as feed supplements to help livestock and poultry grow faster since Jan. 1, 2017. However, due to the increase in the global population, livestock or poultry production output must be increased significantly, and thus there is an unmet need to develop non-antibiotic agents as an alternative approach to the conventional AGP in the livestock industry.

### SUMMARY

In at least one aspect, the present disclosure relates to a method of promoting growth in an animal in need thereof, comprising administering an animal feed composition comprising an effective amount of a feed supplement to the animal, wherein the feed supplement comprises Crassocephalum rabens (C. rabens) or an extract thereof to thereby promote growth of the animal.

In at least one embodiment of the present application, the method comprises administering the animal feed supplement comprising C. rabens or an extract thereof in combination with an additional herbal ingredient. In at least one embodiment of the present application, the additional herbal ingredient is mint essential oil.

In at least one embodiment of the present application, promoting the growth comprises modulating gut microbiota in the animal. In at least one embodiment, modulating the gut microbiota comprises inhibiting or decreasing an amount of pathogenic gut microbiota in the animal. In some embodiments, the pathogenic gut microbiota is at least one selected from the group consisting of Clostridium spp., Streptococcus spp., Veillonella spp., Actinobacillus spp., Escherichia-Shigella, Trueperella spp., Pseudomonas spp., Staphylococcus spp., Enterococcus spp., Pasteurella spp., Helicobacter spp., Lawsonia spp., Mycoplasma spp., and Haemophilus spp. In some embodiments, modulating gut microbiota comprises enhancing or increasing an amount of probiotics microbiota in the animal. In at least one embodiment, the probiotics microbiota is at least one selected from the group consisting of Lactobacillus spp., Megamonas spp., Ruminococcaceae family, Lachnospiraceae family, Ruminococcus_1, Ruminococcus_2, Megamonas spp., Clostridium butyricum, Bacteroides spp., Anaerostipes spp., and Alistipes spp.

In at least one embodiment of the present application, promoting the growth comprises inhibiting or decreasing occurrence of at least one of diarrhea and enteric infection in the animal. In some embodiments, the diarrhea and enteric infection in the animal is associated with at least one bacterial pathogen selected from the group consisting of Escherichia coli, Salmonella spp., Clostridium perfringens, Campylobacter spp., Brachyspira spp., Yersinia spp., Lawsonia spp. and Helicobacter spp.

In at least one embodiment of the present application, the animal feed composition promotes survival of the animal. In at least one embodiment of the present application, the animal feed composition enhances anti-inflammatory response in an animal. In at least one embodiment of the present application, the animal feed composition lowers at least one of TNF-α level and fecal IgA level, or a combination thereof. In some embodiments, promoting the growth comprises increasing bodyweight gain of the animal. In some embodiments, promoting the growth comprises increasing feed efficiency or decreasing feed conversion rate. In some embodiments, promoting the growth comprises modulating primary metabolism in the animal. In at least one embodiment, modulating the primary metabolism comprises increasing or decreasing at least a level of an amino acid, a level of a fatty acid, a level of a carbohydrate, and a level of an organic acid or any combination thereof.

In at least one embodiment of the present application, the animal is a weaned pig. In some embodiments, the animal is a grown-up pig.

In at least one embodiment of the present application, the Crassocephalum rabens or the Crassocephalum rabens extract is in a powder form. In some embodiments, the Crassocephalum rabens serves as a feed additive.

In at least one embodiment of the present application, the Crassocephalum rabens ranges from about 0.001 wt % to about 5 wt % of the animal feed composition, e.g., about 0.001 wt %, about 0.002 wt %, about 0.003 wt %, about 0.004 wt %, about 0.005 wt %, about 0.006 wt %, about 0.007 wt %, about 0.008 wt %, about 0.009 wt %, about 0.01 wt %, about 0.015 wt %, about 0.02 wt %, about 0.03 wt %, about 0.04 wt %, about 0.05 wt %, about 0.1 wt %, about 0.2 wt %, about 0.5 wt %, about 1 wt %, about 1.2 wt %, about 1.5 wt %, about 2 wt %, about 2.5 wt %, about 3 wt %, about 3.5 wt %, about 4 wt %, about 4.5 wt % and about 5 wt %.

In another aspect, the present disclosure relates to a method of preventing or treating a disease caused by porcine reproductive and respiratory syndrome virus (PRRSV) in an animal in need thereof, comprising administering an animal feed composition comprising an effective amount of a feed supplement to the animal, wherein the feed supplement comprises Crassocephalum rabens or an extract thereof. In at least one embodiment, the method comprises administering the animal feed supplement comprising C. rabens or an extract thereof in combination with an additional herbal ingredient. In at least one embodiment of the present application, the additional herbal ingredient may be mint essential oil. In at least one embodiment, the animal feed composition promotes survival of the animal.

In at least one aspect, the present disclosure relates to an animal feed composition comprising a feed supplement including Crassocephalum rabens or an extract thereof. In at least one embodiment of the present disclosure, the Crassocephalum rabens or the Crassocephalum rabens extract is in a powder form. In at least one embodiment, the animal feed composition further comprises an additional herbal ingredient. In at least one embodiment, the herbal ingredient may be mint essential oil. In some embodiments, the animal feed composition comprises a feed supplement comprising Crassocephalum rabens, wherein the Crassocephalum rabens ranges from about 0.001 wt % to about 5 wt % of the animal feed composition, e.g., about 0.001 wt %, about 0.002 wt %, about 0.003 wt %, about 0.004 wt %, about 0.005 wt %, about 0.006 wt %, about 0.007 wt %, about 0.008 wt %, about 0.009 wt %, about 0.01 wt %, about 0.015 wt %, about 0.02 wt %, about 0.03 wt %, about 0.04 wt %, about 0.05 wt %, about 0.1 wt %, about 0.2 wt %, about 0.5 wt %, about 1 wt %, about 1.2 wt %, about 1.5 wt %, about 2 wt %, about 2.5 wt %, about 3 wt %, about 3.5 wt %, about 4 wt %, about 4.5 wt % and about 5 wt %.

These and other aspects will become apparent from the following description of the embodiments taken in conjunction with the drawings, although variations and modifications therein may be affected without departing from the scope of the present disclosure.

The accompanying drawings illustrate one or more embodiments of the disclosure and, together with the written description, serve to explain the principles of the disclosure. Wherever possible, the same reference numbers are used throughout the drawings to refer to the same or like elements of an embodiment.

### BRIEF DESCRIPTION OF THE DRAWINGS

FIGS. 1A to 1G show the effect of Crassocephalum rabens (C. rabens) on growth performance of weaned pigs. Three groups of 4-week-old weaned pigs were fed with standard diet (CTL), standard diet containing low dose (0.25%, LCR) or high dose (0.45%, HCR) C. rabens (CR) powder for 7 weeks, respectively. FIG. 1A are the photographs to show the body phenotypes and shapes of the three groups of pigs. FIG. 1B shows the body weight (kg). FIG. 1C shows the changes of body weight (%). FIG. 1D shows total gain of body weight (kg/pig). FIG. 1E shows feed consumption (kg/pig), and feed conversion ratio (FCR) is shown in FIG. 1F. FIG. 1G shows non-diarrheal incidence/survival rate of the representative piglets from the three groups obtained at the end of feeding experiments. Initial numbers of pigs were 32, 32, and 32; final numbers were 27, 22, and 29 in the CTL, LCR, and HCR groups, respectively. Data are mean±SD. ANOVA was used to compare the difference between control and treatment groups. Asterisk denotes the statistical significance between two compared groups with P<0.05. Different letters (a to c) denote statistical significance (P<0.05) between the three compared groups. Gain of body weight=body weight of the last weighting day−body weight of the first day. Feed conversion ratio (FCR)=total feed consumption quantity/total gain of body weight.

FIGS. 2A to 2D show the effect of C. rabens on feces microbiota of weaned pigs fed with control or CR diets. Rarefaction curves based on rarefied number of bacterial taxa (alternatively Operational Taxonomic Units, OTU) from the feces of the representative weaned piglets from the three groups treated for 7 weeks are shown in FIG. 2A, taxonomic classification of the 16S rRNA gene sequences at the phylum level are shown in FIG. 2B, at the family level as shown in FIG. 2C, and at the genus level as shown in FIG. 2D. Three groups of 4-week-old weaned pigs were fed with standard diet (CTL) and standard diet containing low dose (0.25%, LCR) or high dose (0.45%, HCR) C. rabens (CR) powder for 7 weeks, respectively. Feces samples (n=3) of each group were from 3 pigs, and each value is mean. The bacterial genus is labeled according to their OTUs.

FIGS. 3A to 3E show comparative bacterial 16S rRNA sequences in feces of three groups of weaned pigs fed with control (CTL) or CR diets (LCR or HCR) for 7 weeks, respectively. Score plot is shown in FIG. 3A, corresponding loading plot is shown in FIG. 3B, heatmap is shown in FIG. 3C, pathogen heatmap is shown in FIG. 3D, and probiotic heatmap is shown in FIG. 3E. Three groups of 4-week-old weaned pigs were fed with standard diet (CTL) and standard diet containing low dose (0.25%, LCR) or high dose (0.45%, HCR) C. rabens (CR) powder for 7 weeks, respectively. Feces samples (n=3) of each group were from 3 different pigs, and each value is mean. The bacterial genus is labeled according to their OTUs.

FIGS. 4A to 4P show the effect of C. rabens on pathogen bacterial genera in the feces of weaned pigs fed with control or CR diets. Abundance of total pathogen is shown in FIG. 4A, Clostridium spp. is shown in FIG. 4B, Streptococcus spp. is shown in FIG. 4C, Veillonella spp. is shown in FIG. 4D, Campylobacter spp. is shown in FIG. 4E, Actinobacillus spp. is shown in FIG. 4F, Escherichia Shigella is shown in FIG. 4G, Trueperella spp. is shown in FIG. 4H, Pseudomonas spp. is shown in FIG. 4I, Staphylococcus spp. is shown in FIG. 4J, Enterococcus spp. is shown in FIG. 4K, Pasteurella spp. is shown in FIG. 4L, Helicobacter spp. is shown in FIG. 4M, Lawsonia spp. is shown in FIG. 4N, Mycoplasma spp. is shown in FIG. 4O, and Haemophilus spp. is shown in FIG. 4P. Three groups of 4-week-old weaned pigs were fed with standard diet (CTL) and standard diet containing low dose (0.25%, LCR) or high dose (0.45%, HCR) C. rabens (CR) powder for 7 weeks, respectively. Feces samples (n=3) of each group were from 3 pigs, and each value is mean±SD. ANOVA was used to compare the difference between control and each treatment in the end of the 7th week. Different letters (a to c) denote significant differences between the three groups (P<0.05).

FIGS. 5A to 5L show the effect of C. rabens on probiotic bacterial genera in the feces of weaned pigs fed with control or CR diets. Three groups of 4-week-old weaned pigs were fed with standard diet (CTL) and standard diet containing low dose (0.25%, LCR) or high dose (0.45%, HCR) C. rabens (CR) powder for 7 weeks, respectively. Abundance of total probiotic in the feces of weaned pigs after being fed with CR is shown in FIG. 5A, Lactobacillus spp. is shown in FIG. 5B, Ruminococcaceae family is shown in FIG. 5C, Megasphaera spp. is shown in FIG. 5D, Lachnospiraceae family is shown in FIG. 5E, Ruminococcus 1 is shown in FIG. 5F, Ruminococcus 2 is shown in FIG. 5G, Anaerostipes spp. is shown in FIG. 5H, Clostridium butyricum is shown in FIG. 5I, Megamonas spp. is shown in FIG. 5J, Alistipes is shown in FIG. 5K, and Bacteroides spp. is shown in FIG. 5L. Feces samples (n=3) of each group were from 3 pigs, and each value is mean±SD. ANOVA was used to compare the difference between control and each treatment in the end of the 7th week. Different letters (a to c) denote significant differences between the three groups (P<0.05).

FIGS. 6A and 6B show the effect of C. rabens on fecal IgA concentration as shown in FIG. 6A and on serum TNF-α concentration as shown in FIG. 6B of weaned pigs fed with control or CR diets. Three groups of 4-week-old weaned pigs were fed with standard diet (CTL) and standard diet containing low dose (0.25%, LCR) or high dose (0.45%, HCR) C. rabens (CR) powder for 7 weeks, respectively. Feces and serum samples (n=4) of each group were from 4 pigs, and each value is mean±SD. ANOVA was used to compare the difference between control and each treatment in the end of the 7th week. Different letters (a to c) denote significant differences between the three compared groups (P<0.05).

FIGS. 7A to 7C-2 show the effect of C. rabens on primary metabolites of weaned pig fed with control or CR diets. GC/Q-TOF MS data from CR-fed pig serum analyzed by PLS-DA are shown, score plot is shown in FIG. 7A and corresponding loading plot is shown in FIG. 7B. FIGS. 7C-1 and 7C-2 show the heatmap of the fold change of primary metabolites from CR-fed pig sera compared to the CTL group.

FIG. 8 shows the correlation between probiotics and carbohydrates in weaned pigs fed with control or CR diets with a heatmap of Spearman's rank correlation coefficient between carbohydrate-related probiotics and carbohydrate metabolites (n=3 in each group).

FIGS. 9A to 9H show the effect of C. rabens extract (CRE) and CRE mixed with mint essential oil (CREV) compared to Digestoram (Biomin, Austria) as feed supplement on growth performance and survival rate of weaned pigs. Weaned pigs (5-week-old) were divided into four groups, i.e., animals fed with standard diet (CTL), standard diet plus 0.05% Digestoram (as the positive control), standard diet plus 0.04% CRE, and standard diet plus 0.04% CRE and 0.005% mint essential oil (CREV), respectively. Data are collected during and at the end of feeding experiment. Clinical body shape/size and the physical conditions of piglets are shown in FIG. 9A, body weight (kg/pig) is shown in FIG. 9B, change of body weight (%) is shown in FIG. 9C, total gain of body weight (kg/pig) is shown in FIG. 9D, feed consumption (kg/pig) is shown in FIG. 9E, feed conversion ratio is shown in FIG. 9F, survival rate monitored daily for 6 weeks is shown in FIG. 9G, and serum TNF-α concentrations are shown in FIG. 9H. Initial number of weaned pigs was 32 in each group. Serum samples (n=10) from each group were subjected to determination of TNF-α levels. Data are presented as mean±SD. ANOVA was used to compare the difference within control and all treatment groups. Different letters denote significant differences with P<0.05.

### DETAILED DESCRIPTION

Crassocephalum rabens S. Moore (C. rabens, CR), also named C. crepidioides S. Moore, is a wild vegetable popularly used as a folk medicine in Taiwan for various inflammation-related syndromes. It has been demonstrated that a major galactolipid from CR, 1,2-di-O-α-linolenoyl-3-O-β-galactopyranosyl-sn-glycerol (dLGG), shows preventive and therapeutic effect against sepsis in mouse induced by endotoxin or cecal ligation puncture (Maria K. Apaya, et al. “Simvastatin and a plant galactolipid protect animals from septic shock by regulating oxylipin mediator dynamics through the MAPK-cPLA2 signaling pathway,” Molecular Medicine 2015(21): 988-1001). Enriched galactolipid fraction or dLGG also reveals suppressive effects on melanoma and breast cancer cell metastasis in vitro and in xenograft or allograft mouse tumor models (Chung-Chih Yang, et al., “Plant galactolipid dLGG suppresses lung metastasis of melanoma through deregulating TNF-α-mediated pulmonary vascular permeability and circulating oxylipin dynamics in mice,” International Journal of Cancer 2018(143): 3248-3264; Maria K. Apaya, et al., “Deregulating the CYP2C19/epoxy-eicosatrienoic acid-associated FABP4/FABP5 signaling network as a therapeutic approach for metastatic triple-negative breast cancer,” Cancers (Basel) 2020(12): 199).

In this disclosure, CR for use as a feed additive in weaned pigs is provided. The present disclosure shows that use of CR as a feed additive in weaned pigs decreases diarrhea occurrence, and improves growth performance and survival rate when compared to weaned pigs fed with a control diet. Reprogramming gut microbiota, i.e., increase in the probiotic populations and decrease in pathogenic populations in fecal bacterial communities functioning as probiotics, attenuated IgA levels in pig feces, and modulation of primary metabolism were found to be a relevant action of CR in improving feed efficiency and disease prevention in weaned pigs. Combination of CR extract with mint essential oils (CREV) or CR extract (CRE) alone significantly reduces diarrhea occurrence, improves growth performance and survival rate of treated weaned pigs when compared to the pigs in the control diet group, or the control diet group supplemented with a commercial phytogenics product Digestoram®, which claims to support digestion and overall performance of farm animals.

To develop antibiotic-free AGP for piglets, the first step is to understand microbial and functional succession of the piglet gut microbiome due to its mechanism of altering gut microbial population composition (Geon Goo Han, et al. “Evaluating the association between body weight and the intestinal microbiota of weaned piglets via 16S rRNA sequencing,” Applied Microbiology Biotechnology 2017 (101): 5903-5911; Hyeun Bum Kim, et al., “Microbial shifts in the swine distal gut in response to the treatment with antimicrobial growth promoter, tylosin,” Proceedings of the National Academy of Sciences of the United States of America 2012 (109): 15485-15490). It is crucial to find novel approaches to develop alternatives mimicking the action of AGPs. However, the functional and interactive effects of AGPs on the host and the microbiota are still not well understood. It is therefore desirable to optimize animal health by host and microbial attributes.

In the present disclosure, it is found that a phytogenic additive supports animal health and prevents diseases through modulating piglet gut microbiome.

An animal feed refers to food given to domestic livestock, and pet (companion animal) food.

The term “treatment” refers to use of an effective agent to a subject in need thereof with the purpose to cure, alleviate, relieve, remedy, ameliorate, reduce, or prevent the disease, the symptoms thereof, or the predispositions towards it.

Without intent to limit the scope of the disclosure, exemplary instruments, methods and their related results according to the embodiments of the present disclosure are given below. It is noted that titles or subtitles may be used in the examples for convenience of a reader, which in no way should limit the scope of the disclosure. Moreover, certain theories are proposed and disclosed herein; however, in no way they, whether they are right or wrong, should limit the scope of the disclosure so long as the disclosure is practiced according to the disclosure without regard for any particular theory or scheme of action.

### Materials and Methods

Crassocephalum rabens Plant Powder (CR) and CR Extracts (CRE)

C. rabens plants were harvested, dried and ground by conventional methods known to a skilled person in the art to obtain the plant powder. Both the entire plant and the partial plant without roots may be used to obtain plant powder in this disclosure. C. rabens plant extracts were obtained by freshly pressing of the plants or prepared by 50% to 95% ethanol extraction. The collected extracts in liquid form can be lyophilized or further mixed with additional phytogenic ingredients such as zeolite, pea powder, maltodextrin, wheat bran, or wheat middlings to form an effective formula containing an amount of CR or CRE alone, or a formula containing CR or CRE mixed with other phytogenic ingredients.

### Mint Essential Oils

Mint essential oils can be prepared from varied Mentha species, such as M. spicata ‘Julia's Sweet Citrus’, M. aquativa var. Citrate Lime Mint, M. aquatic var. Kenting Water Mint. In at least one embodiment, mint essential oils were collected from the mint plants cultivated and collected under optimal cultivation conditions in farm fields at Taichung District Agriculture Research and Extension Station, at Chang-Hwa county area or Nantou county area, Taiwan. Matured plants were harvested and subjected to water vapor distillation to collect essential oils. Mint essential oils from different sources can be used alone or mixed with equal volume together to meet the final dose.

### Animals

A total of 96 healthy castrated weaning piglets (Duroc×[Landrace×Yorkshire], initial body weights 7.86±1.25 kg; 28±2 days of age) were obtained from Livestock Business Division, Taiwan Sugar Corporation (Tainan, Taiwan). The piglets were randomly divided into 6 pens with 16 piglets in each pen. According to a completely randomized design, the piglets were allotted to 3 groups assigned into 6 pens, i.e., two pens for each group.

The three weaned piglet groups were: (1) control (CTL) group fed with basal diets based on corn, full-fat soybean meal and whey powder; (2) low-dose (LCR) group fed with basal diets mixed with 0.15% by weight of CR plant powder; and (3) high-dose (HCR) group fed with basal diets mixed with 0.45% by weight of CR plant powder. All diets met the nutrient requirements recommended by the Nutrient Requirements Council (NRC) (1998). The control diet or CR-containing diets (LCR or HCR) were fed for 7 weeks to animals that had already consumed creep feed for three weeks.

In another batch of experiment, the piglets were randomly allocated to 4 groups assigned into 8 pens, two pens for each group. Weaned piglets were divided into (1) CTL group: animals fed with basal diet; (2) Digestoram group: animals fed with basal diets mixed with 0.05% Digestoram (Biomin, Austria), as the positive control group; (3) CRE group: animals fed with basal diets mixed with 0.04% by weight of CR plant extract coating with zeolite; (4) CREV group: animals fed with basal diet plus 0.04% by weight of CRE and 0.005% by weight of mint essential oils. The control diet or control diet plus other formula were supplied to weaned piglets for four weeks (from the animals at age 7-week-old to 11-week-old).

No antibiotics were administered to the experimental piglets throughout the two batches of experiments. The health of all animals was monitored following a previously described method (Stephen G. Matthews, et al., “Early detection of health and welfare compromises through automated detection of behavioral changes in pigs,” The Veterinary Journal 2016 (217): 43-51) and housed in a temperature-controlled nursery room (25±2° C.). Feed and water were available ad libitum. Feed intake and body weight were measured at the end of each week to determine gain of body weight, feed consumption and feed conversion rate (FCR).

During the experiment, all pigs were monitored daily for clinical diarrheal observations. If watery feces were observed, they were classified as either pasty or fluid and recorded as a diarrheal case. The non-diarrheal incidence (%) was calculated as the total number of non-diarrheal piglets over a period divided by the number of piglets in the period multiplied by 100. All animal experiments were performed according to the Animal Care Guidelines of Academia Sinica, Taiwan. All procedures were approved by the Institutional Animal Care and Use Committee of Academia Sinica (protocol no. 19-099-1346) and followed the guidance for the Use of Laboratory Animals (National Academy Press, Washington, DC).

### Feces Sampling

Fecal samples were collected from the anus of weaned pigs just prior to weaning (28 days of age), and 7 weeks after feeding with a standard diet or standard diet plus CR (at 11 weeks of age). The fecal samples were placed in BD BBL™ CultureSwab Plus collection tubes and stored at −80° C.

### Blood Sampling

Whole blood was sampled from the jugular vein in the morning before feed distribution and subjected to hematological examination. Blood samples for biochemical and metabolic profile determinations were drawn in Li-heparin-treated tubes, cooled immediately, and centrifuged for serum separation within 2 hours (h) after collection. Serum was frozen at −20° C. until analyzed. Sera glucose (GLU), total cholesterol (TCHO), triglycerides (TG), blood urea nitrogen (BUN), urea (UA), creatinine (CRE), albumin (ALB), total protein (TP) were determined. Alanine aminotransferase (ALT) and aspartate aminotransferase (AST) were used to assess hepatocellular function. Alkaline phosphatase (ALP) was considered both for its possible involvement in description of hepatobiliary injury and as a marker of osteoblast activity. Total bilirubin was used as an index of hepatobiliary injury.

Analysis of composition of intestinal microflora Genomic DNA (gDNA) of the fecal samples was extracted by commercial DNA extraction kit (QIAamp Powerfecal DNA Kit, QIAGEN, Hilden, Germany). gDNA concentration was determined and adjusted to 5 ng/μg for PCR amplification reaction and purification. For the 16S rRNA gene sequencing, the V3-V4 region was amplified by a specific primer set (319F: 5′-CCTACGGGNGGCWGCAG-3′ (SEQ ID NO: 1); and 806R: 5′-GACTACHVGGGTATCTAATCC-3′ (SEQ ID NO: 2)) (N=A, T, C or G; W=A or T; H=A, C, or T; V=A, C, or G) according to the 16S Metagenomic Sequencing Library Preparation procedure (Illumina). In brief, 5 μg of gDNA was used for the PCR reaction carried out with KAPA HiFi HotStart Ready Mix (Roche) under the PCR condition: 95° C. for 30 seconds (s); 55° C. for 30 s; 72° C. for 30 s; 72° C. for 5 min; and then held at 4° C. The PCR products were examined on a 1.5% agarose gel. Samples with a bright major band around 500 bp were chosen and purified by using the AMPure XP beads for the subsequent library preparation.

The library for sequencing was prepared according to the 16S Metagenomic Sequencing Library Preparation procedure (Illumina). In brief, a secondary PCR was performed by using the 16S rRNA V3-V4 region PCR amplicon and Nextera XT Index Kit with dual indices and Illumina sequencing adapters (Illumina). The indexed PCR product quality was assessed on the Qubit 4.0 Fluorometer (Thermo Scientific) and Qsep100™ system. The results of 16S rRNA gene amplicons sequencing were analyzed by MiSeq Reporter, BaseSpace and Greengenes database for taxonomy and classification of microbiota between CR-treated or non-treated swine.

Detection of Porcine Reproductive and Respiratory Syndrome Virus (PRRSV) in Saliva Samples from Weaned Pigs

RNAs were extracted from saliva samples with the QIAamp cador Pathogen Mini Kit (QIAGEN GmbH, Hilden, Germany) following the kit instructions ready for PCR experiment. PCR was carried out by using a real-time multiplex RT-PCR Test Kit (QIAGEN GmbH, Hilden, Germany) for detection of European (EU) genotype, North American (NA) genotype, and highly pathogenic NA strain (HP) of PRRS viruses (Labor Diagnostik GmbH Leipzig) containing enzymes, primers and probes in one mixture (PRRSV-Mix), and a positive and a negative control. The test kit can be used to detect EU- and NA-genotype of PRRSV, a HP strain of NA-genotype and an amplification- and extraction control (mRNA of 3-actin housekeeping gene) at the same time. PRRSV-Mix was prepared with RNA elution using Optical Tube Strips (Agilent Technologies). The formulation per sample included 80% of PRRSV-Mix and 20% of sample or controls. PCR was carried out on an ABI 7500 Real-time PCR system (Applied Biosystems) using the following thermal profile: reverse transcription reaction at 45° C. for 10 min; initial PCR activation step at 95° C. for 10 min; 40 cycles of denaturation at 95° C. for 15 s; annealing at 56° C. for 30 s; and extension at 72° C. for 30 s. All samples were tested in duplicate.

### Analysis of Fecal IgA and Seral Tumor Necrosis Factor-α (TNF-α) in Weaned Pigs

Fecal IgA was determined by pig IgA ELISA kit (Bethyl Laboratories, Montgomery, TX, USA). Fresh feces samples and controls were diluted at 1:10 w/v (0.1 g in 0.9 mL) with assay diluent, thoroughly homogenized by vortex for 30 s and centrifuged at 4000×g for 10 min. All samples were tested in triplicate. The relative IgA antibody level was quantified using a standard curve made using two-fold serial dilutions of a positive reference. The interpolation from the standard curve employed non-linear regression with least square fits using Graphpad Prism 5.0 (GraphPad Software, San Diego, CA, USA) measured at 450 nm. The concentration of TNF-α in serum was determined by commercial enzyme-linked immunosorbent assay (ELISA) kit (R&D Systems, USA) according to the manufacturer's instructions.

### Primary Metabolome Analysis Using Gas Chromatography/Quadrupole Time-of-Flight Mass Spectrometry (GC/Q TOF-MS)

The serum samples (50 μL each) from tested weaned pigs were mixed with 80% methanol containing ribitol (0.2 mg/mL) as the internal standard, then vigorously vortexed and put in liquid nitrogen for 10 min. The protocol was repeated 3 times to thoroughly remove the protein fraction. After centrifuging at 13,000 rpm for 10 min at 4° C., the supernatants were collected and dried in a vacuum by SpeedVac (Labconco, USA). The dried analytes were incubated with 20 μL methoxyamine (20 mg/mL in pyridine) at 30° C. for 90 min, and then reacted and derivatized with 100 μL N, O-bis(trimethylsilyl)trifluoroacetamide (BSTFA) containing 1% trimethylchlorosilane (TMCS) at 70° C. for 120 min.

Investigation of the primary metabolome of pig serum samples was performed using a GC/Q-TOF mass spectrometer (Agilent Technologies, USA) at the Metabolomics Core Facility, Agricultural Biotechnology Research Center, Academia Sinica, Taiwan. The derivatized samples (0.5 μL) were injected with helium as the carrier gas flow at 1 mL/min into an Agilent J&W DB-5 ms column (30 m×250 μm×0.25 μm). The GC oven temperature ramp was maintained at 60° C. for 1 min, then elevated to 325° C. (10° C./min) and held constant for 10 min. The mass range was 50 to 600 Da, and the data were gathered in a full scan mode. Mass spectra were compared against the NIST Chemistry WebBook (National Institute of Standard and Technology) and PubChem (National Center for Biotechnology Information). Peak heights of the mass (mass-to-charge ratio) fragments were normalized to the internal standard (ribitol) of each sample.

### Data Analysis and Statistics

Multivariate partial least squares discriminant analysis (PLS-DA) was used to define the maximum classification and separation of independent samples. The statistics analysis was performed by the SAS program (SAS Institute, USA). The difference between groups was determined by ANOVA. P<0.05 was considered statistically significant and indicated by different letters.

### Example 1: Effect of CR Plant Powder on Growth Performance in Weaned Pigs

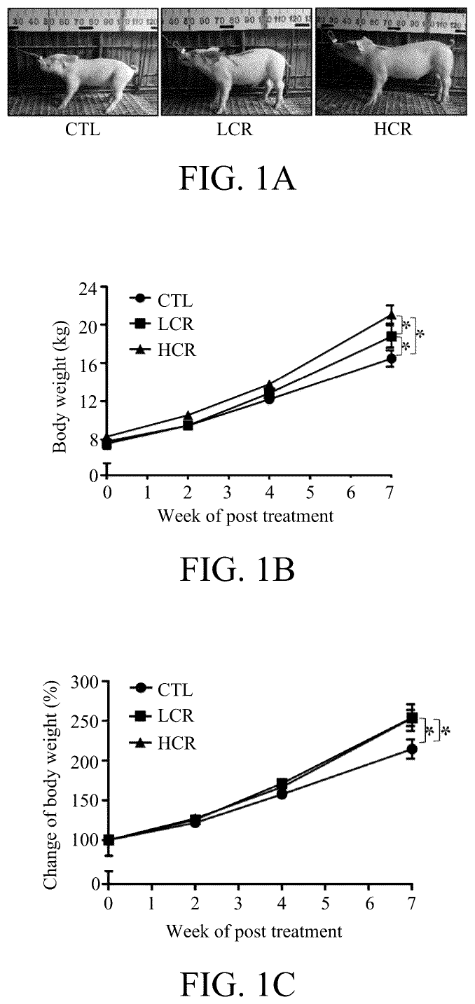

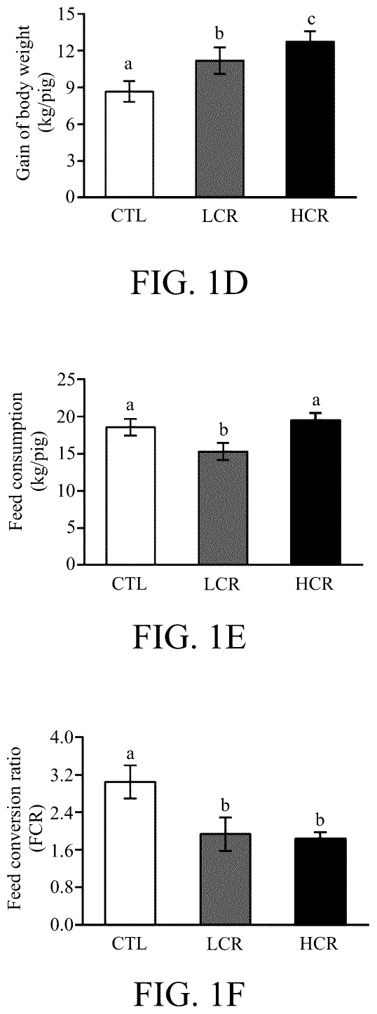

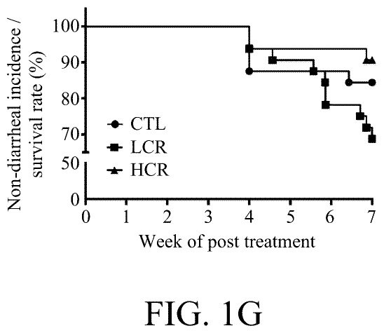

Weaned pigs were fed with basal diet and low-dose or high-dose CR diet for 35 days. Clinical observations, feed consumption and survival rate were recorded daily over the experimental period. The effects of CR supplement on growth performance in weaned pigs are summarized in FIGS. 1A to 1G. Clinical body shape/size and the physical condition of piglets in both the LCR and HCR groups were much better than the CTL group, and the HCR group animals were significantly bigger than the LCR and CTL groups, as shown in FIG. 1A. After 7 weeks of CR treatment, both LCR and HCR groups of weaned pigs exhibited significantly increased body weights in comparison with the CTL group. LCR and HCR weaned piglets had increased their body weight by 13.98% and 27.78%, respectively, compared to the CTL group (P<0.05) at the end of the 7-week treatment period, as shown in FIGS. 1B and 1C and Table 1 below.

TABLE 1
Effect of Crassocephalum rabens plant on the parameter
of body weight and feed conversion rate of weaned pigs

| | CTL | LCR | HCR |
|---|---|---|---|
| Initial weight (28 Days) | 7.79 ± 0.20^{a} | 7.57 ± 0.34^{a} | 8.29 ± 0.19^{b} |
| Final weight (77 Days) | 16.45 ± 0.84^{a} | 18.75 ± 1.16^{b} | 21.02 ± 0.94^{c} |
| Total weight gained in | 8.66 ± 0.87^{a} | 11.18 ± 1.08^{b} | 12.73 ± 0.86^{b} |
| 50 days (kg) | | | |
| Total feed intake in 50 | 18.55 ± 1.14^{a} | 15.24 ± 1.17^{b} | 19.47 ± 0.98^{a} |
| days (kg) | | | |
| Feed conversion rate | 3.05^{a} | 1.93^{b} | 1.84^{b} |
| (FCR) | | | |

Mean ± SEM (number of pigs in CTL, LCR, and HCR groups is 27, 22, and 29, respectively). ANOVA was used to compare the difference between CTL (basal diet), LCR (basal diet + 0.25% CR) and HCR (basal diet + 0.45% CR) groups. Different letters (a, b and c) denote statistical significance (P < 0.05).

Furthermore, the HCR group piglets were on average 2.27 kg heavier (P<0.05) at the end of the nursery period than the LCR group, as shown in Table 1 above. The gain of body weight in the CR treatment groups was significantly higher than the control group, as shown in FIG. 1D. From FIG. 1E, although total feed consumption in the LCR group was lower than the CTL and HCR groups, FCR was significantly enhanced in the LCR (1.93) and HCR (1.84) groups compared to the CTL group (3.05) (P<0.05) as shown in FIG. 1F and Table 1, indicating that CR plant promoted the growth performance of weaned pigs.

Reduction of diarrheal incidence in animals with reference to survival rate of weaned pigs in the three groups is presented in FIG. 1G. Survival rate of pigs fed with HCR (90.63%) was much higher than the CTL group (84.38%) and those fed with LCR (68.75%).

### Example 2: Effect of CR Plant Powder on PRRS Incidence in Weaned Pigs

Porcine reproductive and respiratory syndrome (PRRS) is currently the most economically important disease affecting pig producers. To assess the effect of CR on PRRS incidence in the CTL, LCR and HCR groups of pigs, saliva samples were randomly selected from 12 pigs. During the feed period, saliva samples from 4 pigs were randomly selected from each group for PRRS analysis at the initial day (Day 28) and final day (Day 77). The effects of CR supplement on PRRS incidence in weaned pigs are summarized in Table 2 below. The detected results of all the pigs were negative when they were 28 days old. The PRRS incidence among 77-day-old pigs in the CTL, LCR, and HCR groups was 75%, 50% and 0%, respectively.

TABLE 2
Effect of Crassocephalum rabens on PRRS virus distribution
in weaned pigs fed with control or CR supplemented diets^{a}

| | CTL | LCR | HCR |
|---|---|---|---|
| Initial Day (28 Days) | Non-detected | Non-detected | Non-detected |
| Final Day (77 Days) | 3/4 | 2/4 | Non-detected |
| | 1/4: EU & NA genotype | 2/4: NA genotype | |
| | 1/4: NA genotype & HP | | |
| | strain | | |
| | 1/4: NA genotype | | |

^{a}Results are presented as the number of positive PRRS virus cases/total samples tested (n = 4) in the saliva samples analyzed by RT-PCR method.

### Example 3: Hematological and Biochemical/Physiological Parameters in Weaned Pigs

Peripheral blood cell profiles of weaned pigs fed with control diet or CR-supplemented diets were examined. Analysis of complete blood counts of animals (n=4) in each group were conducted on week 7 with a hemocytometer, as shown in Table 3 below. On average, most of the cell types in the whole blood of the three groups of animals did not show a significant difference and were within the normal range of healthy animals, except that the lymphocyte (77˜80) and WBC (23.9˜30.6) populations were slightly higher in all three groups of animals than the values in a normal range while neutrophils were detected in all three groups of tested animals (18.7˜22.7).

TABLE 3
Hematological parameters in weaned pigs fed with control or CR diets*

| Cell type | CTL | LCR | HCR | References^{1, 2} |
|---|---|---|---|---|
| WBCs (10³/μL) | 23.9 ± 6.7 | 30.6 ± 1.1 | 27.9 ± 5.2 | 6.3~21.1¹ |
| RBCs (10³/μL) | 6.1 ± 0.3 | 6.8 ± 0.2 | 6.4 ± 0.4 | 4.4~8.6¹ |
| Hb (g/dL) | 9.7 ± 0.3 | 10.8 ± 0.2 | 10.1 ± 0.7 | 9.0~16.2¹ |
| HCT (%) | 34.6 ± 1.3 | 38.4 ± 0.5 | 36.9 ± 1.5 | 33.9 ~45.9¹ |
| MCV (fL) | 57.0 ± 2.9 | 56.5 ± 1.9 | 58.1 ± 2.3 | 46.6~77.2² |
| MCH (pg) | 16.0 ± 0.8 | 15.9 ± 0.6 | 15.9 ± 0.6 | 11.2~17.6² |
| MCHC (g/dL) | 28.2 ± 0.3 | 28.2 ± 0.5 | 27.4 ± 0.8 | 19.6~28.0² |
| RDW (%) | 16.5 ± 0.7 | 14.7 ± 0.4 | 15.8 ± 0.9 | — |
| Platelets (10³/μL) | 283.8 ± 97.5 | 295.0 ± 95.6 | 251.0 ± 80.8 | 220~665¹ |
| Neutrophils (%) | 19.50 ± 3.8 | 22.7 ± 5.3 | 18.7 ± 2.9 | — |
| Lymphocytes (%) | 80.50 ± 3.8 | 77.3 ± 5.3 | 80.3 ± 3.3 | 38.1~73.1¹ |
| Monocyte (%) | 0.0 ± 0.0 | 0.0 ± 0.0 | 1.0 ± 0.0 | 0~15.0¹ |
| Eosinophils (%) | 0.0 ± 0.0 | 0.0 ± 0.0 | 0.0 ± 0.0 | 0~7.7¹ |
| Basophils (%) | 0.0 ± 0.0 | 0.0 ± 0.0 | 0.0 ± 0.0 | 0~1.3¹ |

*Serum samples (n = 4) of weaned pigs were collected after 7 weeks of treatment. The data are mean ± SD. The total pig number of the CTL, LCR and HCR groups were 27, 22 and 29, respectively. WBCs, white blood cells; RBCs, red blood cells; Hb, hemoglobin; HCT, hematocrit; MCV, mean corpuscular volume; MCH, mean corpuscular Hb; MCHC, mean corpuscular Hb concentration; RDW, platelet distribution width; Platelets, platelets count.
¹Lee Sung Pigs of National Taiwan University, Department of Animal Science and Technology and Taiwan Agricultural Technology Research Institute.
²Breeding and supply of laboratory minipigs, Livestock Research Institute, Council of Agriculture,Executive Yuan.

Table 4 below shows the mean values of biochemical/physiological parameters in the serum of CTL, LCR and HCR weaned pigs (n=4 in each group). In general, feeding with CR diets did not affect the levels of lipid, glucose, kidney function related markers, liver index enzymes, or varied protein concentrations, such as TG, total cholesterol, blood urea nitrogen, uric acid, creatinine, albumin, aspartate aminotransferase (AST), alanine aminotransferase (ALT), alkaline phosphatase (ALP), among others, indicating that CR supplementation did not have a deleterious or toxic effect on the tested animals.

TABLE 4
Biochemical parameters in the serum of weaned pigs fed with control or CR diets

| Parameters | CTL | LCR | HCR | References^{1, 2} |
|---|---|---|---|---|
| GLU (mg/dL) | 104.4 ± 29.6 | 102.0 ± 16.7 | 87.2 ± 6.0 | 91~173 |
| AST (U/L) | 48.6 ± 14.8 | 40.7 ± 6.6 | 55.2 ± 20.6 | 30~68 |
| ALT (U/L) | 42.6 ± 15.2 | 35.3 ± 6.0 | 39.2 ± 11.4 | 14~62 |
| ALP (U/L) | 372.6 ± 141.2 | 435.0 ± 94.9 | 242.0 ± 81.0 | 207~477 |
| BUN (mg/dL) | 10.5 ± 2.9 | 8.9 ± 0.4 | 10.7 ± 2.7 | 10.8~18.4 |
| CRE (mg/dL) | 0.9 ± 0.1 | 0.9 ± 0.1 | 1.0 ± 0.2 | 0.6~1.4 |
| UA (mg/dL) | 0.4 ± 0.1 | 0.4 ± 0.1 | 0.4 ± 0.0 | <2¹ |
| TCHO (mg/dL) | 71.4 ± 9.9 | 84.7 ± 13.4 | 83.6 ± 9.2 | 76~162 |
| TG (mg/dL) | 71.0 ± 22.8 | 77.0 ± 11.2 | 61.8 ± 31.6 | 29~139 |
| TP (g/dL) | 6.3 ± 0.9 | 5.8 ± 0.4 | 6.8 ± 1.4 | 5.5~7.3 |
| ALB (g/dL) | 2.4 ± 0.4 | 2.6 ± 0.3 | 2.6 ± 0.3 | 1.84 ~2.20 |
| GLOB (g/dL) | 3.9 ± 0.7 | 3.2 ± 0.3 | 4.2 ± 1.3 | 3.6~5.2 |

Serum samples (n = 4) of weaned pigs were collected after 7 weeks of treatment. The data are mean ± SD.The total pig number of the CTL, LCR and HCR groups were 27, 22 and 29, respectively. GLU, glucose; AST, aspartate aminotransferase; ALT, alanine aminotransferase; ALP, alkaline phosphatase; BUN, blood urea nitrogen; CRE, creatinine; UA, uric acid; TCHO, total cholesterol; TG, triglyceride; TP, total protein; ALB, albumin; GLOB, globulin.
¹Lee Sung Pigs of National Taiwan University, Department of Animal Science and Technology and Taiwan Agricultural Technology Research Institute.
²Breeding and supply of laboratory minipigs, Livestock Research Institute, Council of Agriculture, Executive Yuan.

### Example 4: Analysis with 16S rRNA MiSeq Sequencing Data

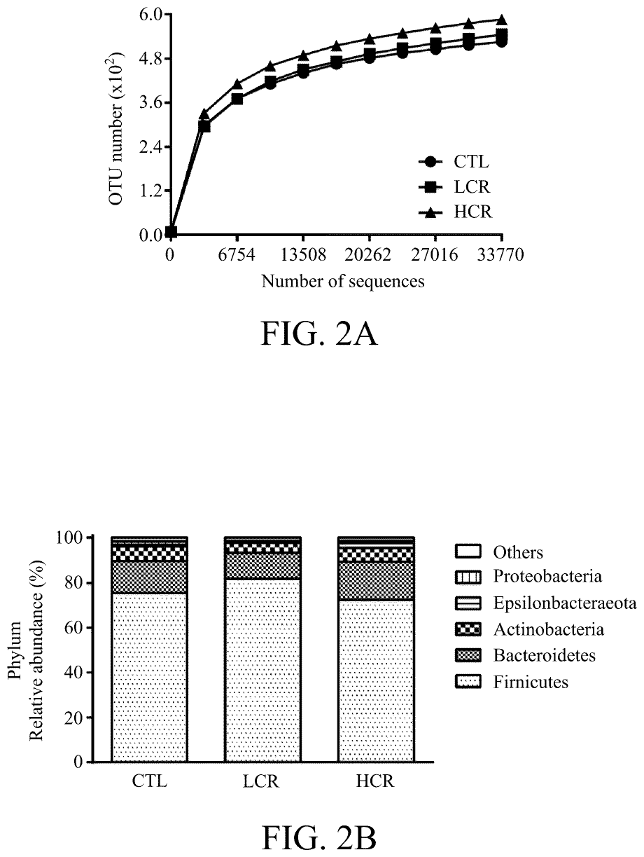

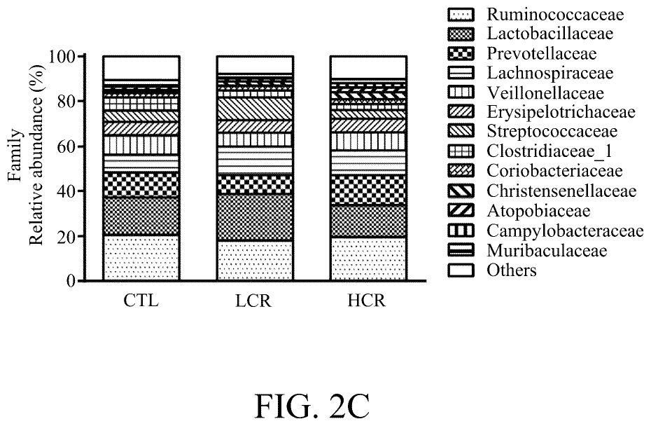

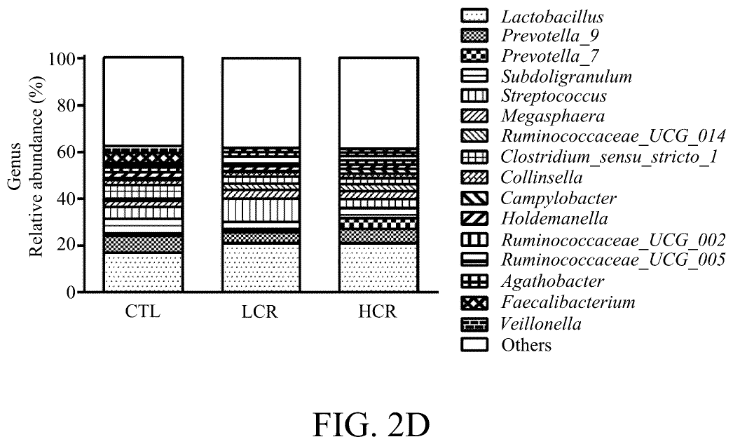

Sequencing of the 16S rRNA genes in the 9 fecal samples produced a total of 289,839 reads after quality-filtering, with a mean sequence number of 96,613±29,482 reads per sample. Evaluation of rarefaction curves based on rarefied number of taxa (alternatively Operational Taxonomic Units, OTU) on sample datasets indicated that all 3 curves (each curve was a mean of 3 pigs) tend to form a plateau which bears evidence of adequate sequence coverage for the majority of biodiversity contained within the samples, as shown in FIG. 2A, which demonstrated that almost all the bacterial species were detected in the colonic microbiota. The mean number of observed OTUs identified in the CTL, LCR, and HCR groups were 525.7±106.1, 545.0±136.1, and 586.7±79.7, respectively, as shown in Table 5 below. Rarefaction curves did not reveal a noticeable difference among bacterial taxa composition in the feces contents of the 9 pigs; slightly higher alpha-diversity index values (PD whole tree, Chao 1, Shannon-Weaver index, and ACE) were noticed in the HCR group, compared to the CTL group without showing statistical significance.

TABLE 5
Alpha diversity of the pig gut microbiota
determined based on 16S rRNA gene sequences

| Diversity index | CTL | LCR | HCR |
|---|---|---|---|
| PD whole tree | 7.93 ± 0.75 | 8.68 ± 1.18 | 8.85 ± 0.48 |
| Shannon | 6.41 ± 0.53 | 6.21 ± 0.78 | 6.67 ± 0.50 |
| Simpson | 0.97 ± 0.01 | 0.96 ± 0.02 | 0.97 ± 0.01 |
| Chao 1 | 590.4 ± 146.2 | 652.1 ± 185.5 | 680.1 ± 87.4 |
| ACE | 585.1 ± 143.1 | 622.8 ± 171.5 | 659.7 ± 95.7 |
| Observed OTUs | 525.7 ± 129.9 | 545.0 ± 166.7 | 586.7 ± 97.7 |

Feces samples (n = 3) of weaned pigs were collected after 7 weeks of treatment. The data are mean ± SD. The total pig number of the CTL, LCR and HCR groups were 27, 22 and 29, respectively. PD whole tree, phylogenetic diversity; Shannon diversity index, estimator of species richness and species evenness: more weight on species richness; Simpson's index, estimator of species richness and species evenness: more weight on species evenness; Chao 1, abundance-based estimator of species richness; ACE, abundance-based coverage estimator of species richness; observed OTUs, estimator of species richness.

### Example 5: Bacterial Diversity in Weaned Pigs and CR Feed Pigs Based on Gene Data

### Comparison of Gut Microbial Communities

To assess the comparative profiling and composition of gut microbiota in the CTL, LCR, and HCR groups of pigs, feces samples were randomly selected from nine pigs (three pigs from each group) and subjected to 16S rRNA MiSeq sequencing. The overall structural changes in gut microbiota in weaned pigs in response to CR were determined. Metagenomic analysis indicated that the gut bacteria in weaned pigs belonged to 25 phyla, 31 classes, 57 orders, 99 families, and 206 genera. OTU-level analyses were performed to determine the phylum as shown in FIG. 2B, family as shown in FIG. 2C, and genus-level as shown in FIG. 2D composition of the bacterial communities using all samples combined.

Assigned taxonomic profiles at the bacterial phylum level for pooled feces samples revealed that in the CTL group, Firmicutes was the dominant phylum (75.5%) followed by Bacteroidetes (14.3%), Actinobacteria (6.4%), and Proteobacteria (1.5%). In the LCR and HCR groups, bacterial phyla distribution was dominated by Firmicutes (81.9 and 72.5%) followed by Bacteroidetes (11.4% and 16.9%), Actinobacteria (4.4% and 6.1%), Epsilonbacteraeota (0.31% and 2.05%), and Proteobacteria (0.5% and 0.9%), as shown in FIG. 2B. The CTL group had the highest relative abundance of Proteobacteria (2.98-fold and 1.78-fold compared to the LCR and HCR groups). It is to be noted that Proteobacteria include a wide variety of pathogens, such as Escherichia, Campylobacter, Salmonella, Helicobacter, Pseudomonas, and many other notable pathogenic genera.

At the family level, the three most abundant bacterial families in the CTL group of weaned pigs' microbiota primarily consisted of Ruminococcaceae (20.9%), Lactobacillaceae (16.6%), and Prevotellaceae (11.0%). The four abundant bacterial families in the LCR group primarily consisted of Lactobacillaceae (20.88%), Ruminococcaceae (17.97%), Lachnospiraceae (12.94%), and Streptococcaceae (10.07%). The HCR group possessed the highest relative abundance of Ruminococcaceae (19.6%), Lactobacillaceae (14.1%), Prevotellaceae (13.5%), and Lachnospiraceae (10.9%), as shown in FIG. 2C. It is to be noted that Ruminococcaceae, Lactobacilliaceae, Prevotellaceae, and Lachnospiraceae are numerous gut microbes with roles in the breakdown of proteins and carbohydrates in foods. The LCR and HCR groups possessed higher relative abundance (59.97% and 58.10%) of these four bacterial families than the CTL group (56.29%).

At the genus level, Lactobacillus was the topmost significantly enriched genera in gut microbiota in the three groups of pigs. The abundance of Lactobacillus in the CTL, LCR and HCR groups were 16.6%, 20.9% and 19.1%, respectively. The genera Ruminococcaceae (6.6% and 6.5%) and Prevotella (5.4% and 10.7%) were more abundant in the LCR and HCR groups, while Prevotella (8.1%), Subdoligranulum (6.1%), and Clostridium (5.8%) were the most abundant genera in the CTL group, as shown in FIG. 2D.

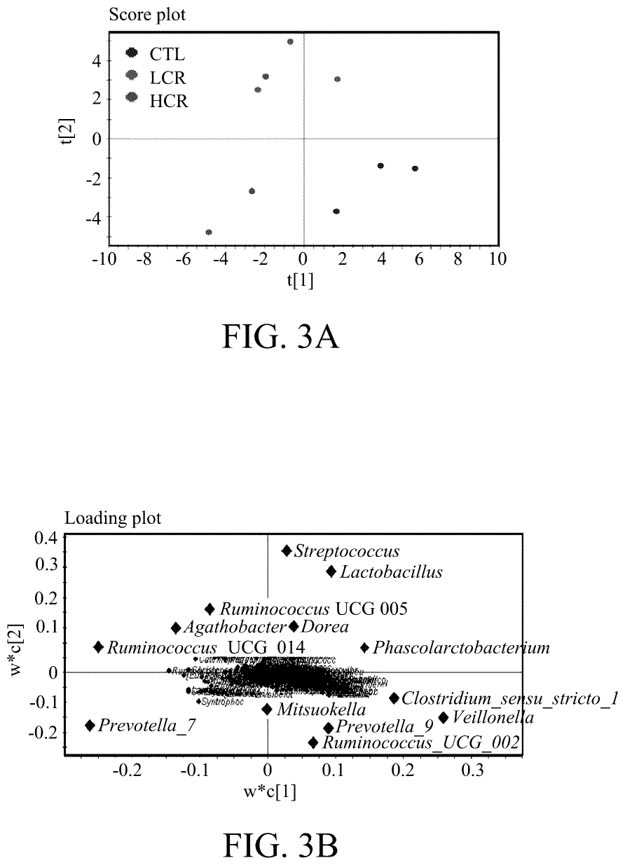

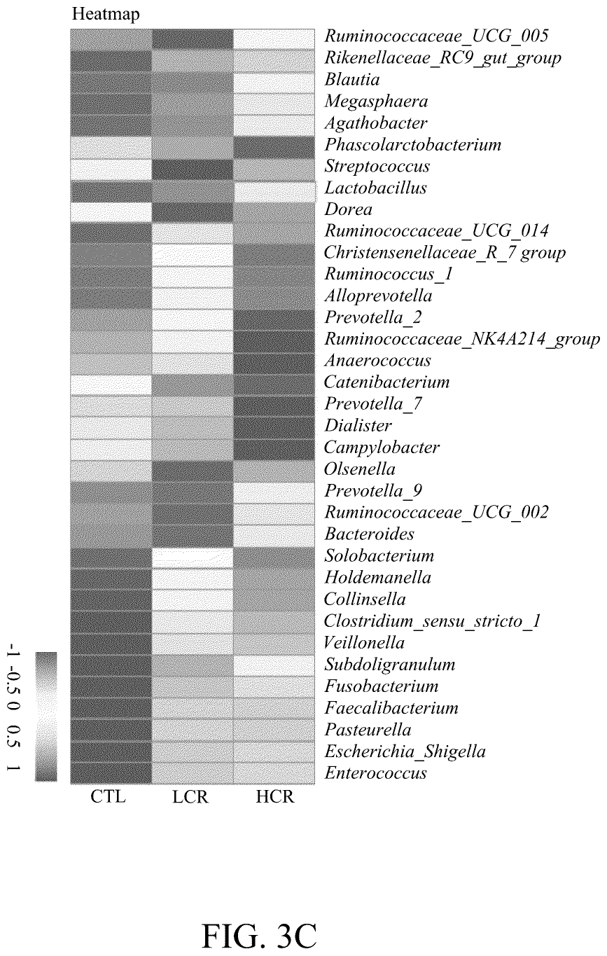

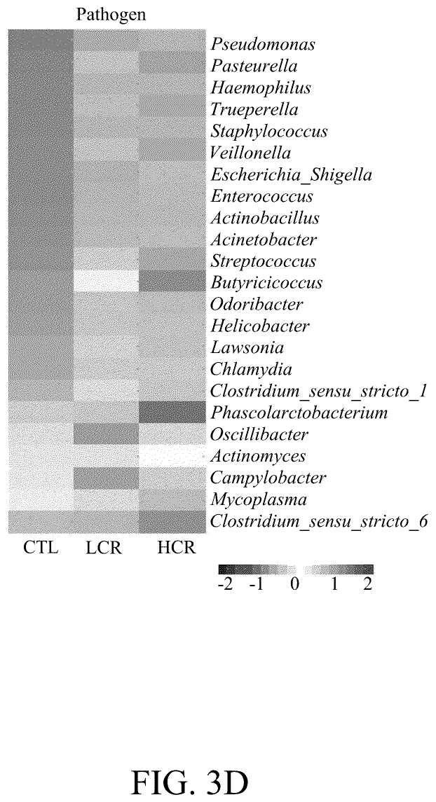

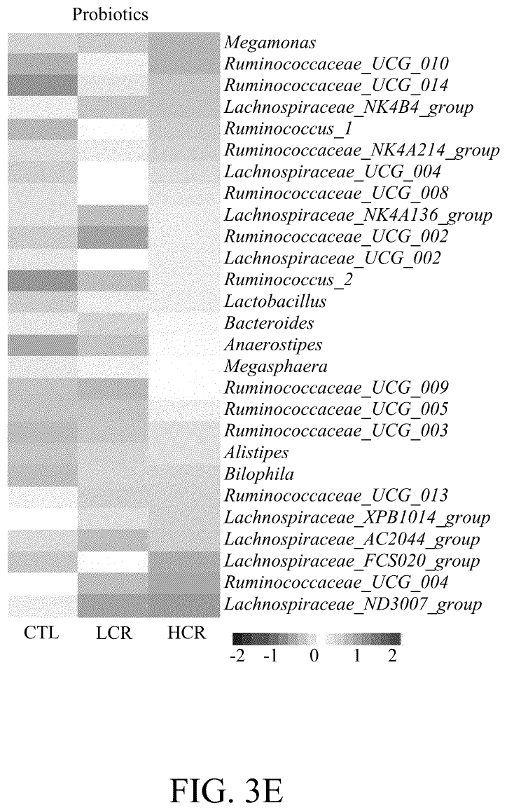

A total of 206 genera detected by 16S rRNA sequencing were subjected to multivariate analysis by partial least squares discriminant analysis (PLS-DA). PLS-DA revealed distinct clustering of intestinal microbe communities for each experimental group. CR induced changes in the community structure of weaned pig fecal microbes as revealed by the PLS-DA score plot, as shown in FIG. 3A. In the score plot, each point represents an individual test pig, and grouping, trends, and outliers can be observed. The score plot reveals distinct clustering of each group, which is an indication that CR treatment was involved in bacterial reprogramming or changes. The corresponding loading plot identifies the outlier microbes that have a strong association to three specific groups of animals, as shown in FIG. 3B. The distance of a genus of microbiota from the origin represents the contribution to the clustering of different groups on PLS-DA. Remarkable changes in the microbiota community structure and specific genera of microbes were induced by both LCR and HCR interventions relative to CTL. Both high and low doses of CR consumption induced similar microbial composition changes.

The differences between genus abundance measurements were further analyzed using Z-score by groups and used to produce a heatmap as shown in FIG. 3C. Some of the relative abundance changes at the genus level were also identified. OTUs in the Solobacterium, Holdemanella, Collinsella, Clostridium_sensu_stricto_1, Veillonella, Subdoligranulum, Fusobacterium, Faecalibacterium, Pasteurella, Escherichia-Shigella, and Enterococcus genera that are highly abundant in the CTL were reduced in the CR groups. Similar differential tendencies were observed in both the LCR and HCR groups of weaned pigs. The most abundant OTUs in the LCR group were Ruminococcaceae_UCG 005, Rikenellaceae_RC9_gut_group, Blautia, Megaspeaera, Agathobacter, Phascolarctobacterium, Streptococcus, Lactobacillus, and Dorea. The dominant OTUs in the HCR group were Ruminococcaceae_UCG_014, Christensenellaceae_R_7_group, Ruminococcus_1, Alloprevotella, Prevotella_2, Ruminococcaceae_NK4A214_group, Anaerococcus, Catenibacterium, Prevotella_7, Dialister, and Campylobacter.

The differences between pathogen and probiotic genera abundance measurements were further analyzed using Z-score by group and used to produce heatmaps as shown in FIG. 3D and FIG. 3E, respectively. With regard to pathogen abundance, it is shown that the CTL group was highest, and the CR group had significantly lower abundance. However, three groups have their own advantage with regard to different probiotic abundance. Megamonas, Ruminococcaceae_UCG_010, Ruminococcaceae_UCG_014, Lachnospiraceae_NK4B4_group, and Ruminococcus_1 were higher in the HCR group; Lachnospiraceae_ND3007_group, Lachnospiraceae_AC2044_group, Ruminococcaceae_UCG_004, Ruminococcus_2, Ruminococcaceae_UCG_005, Anaerostipes, and Ruminococcaceae_UCG_013 were higher in the LCR group; and Ruminococcaceae_UCG_003, Bilophila, Ruminococcaceae_UCG_009, Alistipes, Lachnospiraceae_FCSO20_group, and Ruminococcaceae_UCG_002 were higher in the CTL group.

### Analysis of Pathogenic and Probiotic Communities

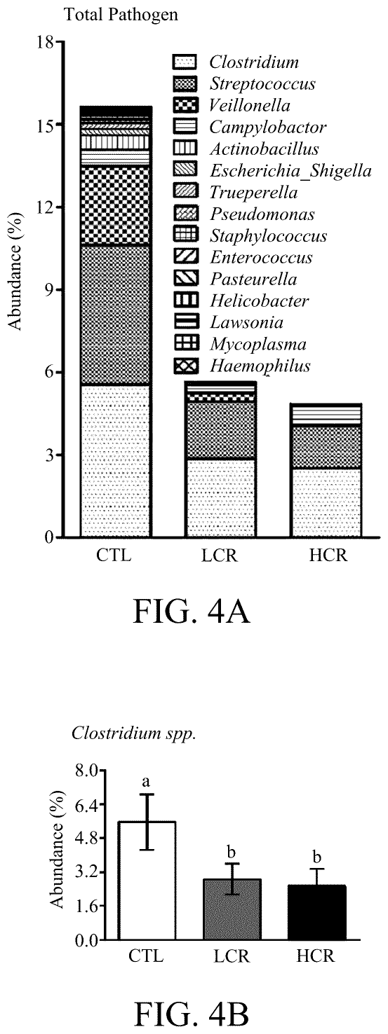

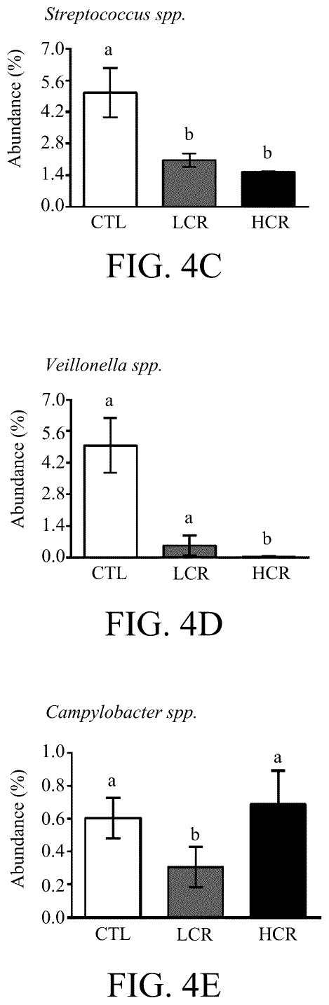

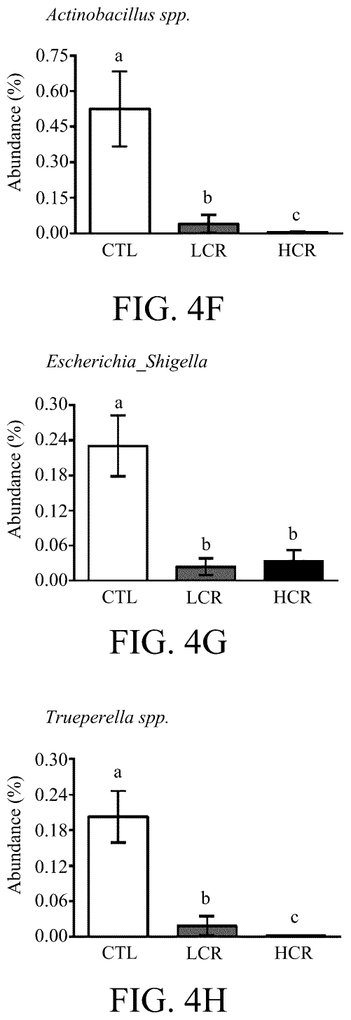

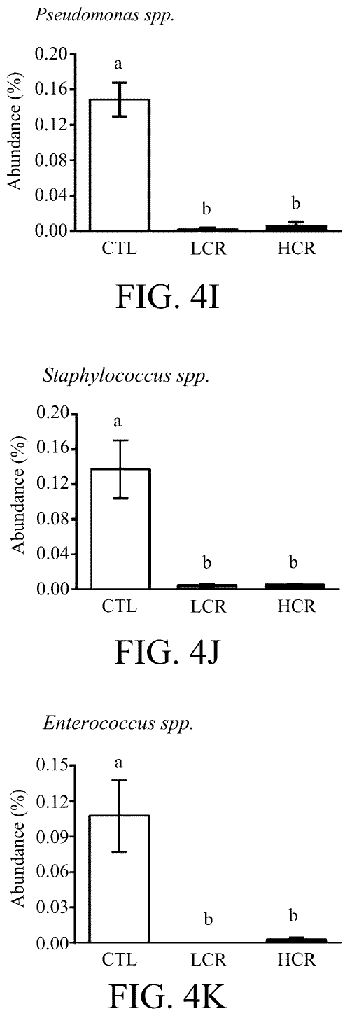

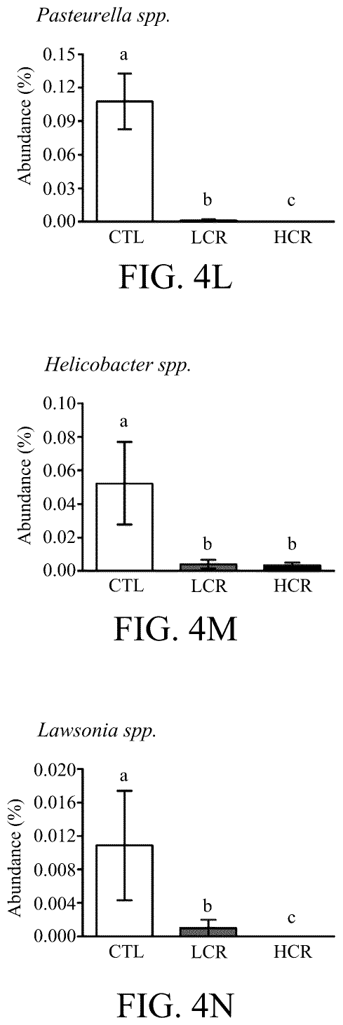

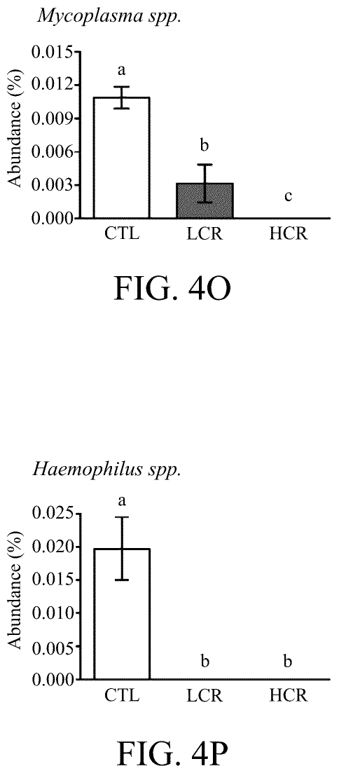

The abundance of pathogenic bacterial genera was analyzed to show the effect of CR on weaned pigs, as shown in FIGS. 4A to 4P.

Bacterial pathogens such as Escherichia Shigella (FIG. 4G), Clostridium perfringens, Campylobacter spp. (FIG. 4E), Lawsonia spp. (FIG. 4N) and Helicobacter spp. (FIG. 4M), which cause diarrhea and porcine gastric ulcers in weaned pigs were analyzed. Abundances of genera Escherichia Shigella were 0.230%, 0.024%, and 0.034% in the CTL, LCR, and HCR groups, respectively. Clostridium spp. is found to present in the CTL group in a significantly higher abundance than in LCR and HCR groups. For example, Clostridium perfringens is found to present only in CTL (0.015%). Campylobacter spp. were less abundant in the LCR group (0.307%) compared to CTL (0.604%) and HCR (0.687%). Lawsonia spp. and Helicobacter spp. were present in the CTL (0.0109% and 0.0523%) group with a relatively much higher abundance than those in the LCR (0.001% and 0.0039%) and HCR (0% and 0.0033%) groups, respectively.

Respiratory lesions can be categorized into three main disease entities: rhinitis, pneumonia, and pleuritis. Related bacterial pathogens of the respiratory tract are known to be Pasteurella spp. (FIG. 4L), Mycoplasma spp. (FIG. 4O), Streptococcus suis, Haemophilus spp. (FIG. 4P), Actinobacillus spp. (FIG. 4F), Trueperella spp. (FIG. 4H), Bordetella spp., and Salmonella spp. The abundance of genera Pasteurella was found to be 0.1076%, 0.0010%, and 0% in CTL, LCR, and HCR, respectively. Mycoplasma spp. were significantly present in the CTL (0.0109%) group compared to the LCR (0.0038%) and HCR (0%) groups. Streptococcus suis was higher in the CTL (0.0059%) group compared to the LCR (0.001%) and HCR (0%) groups, while Haemophilus spp. (FIG. 4P) was only detected in the CTL (0.0197%) group. Actinobacillus spp. (FIG. 4F) was present in relatively high abundance in the CTL (0.5251%) pigs in comparison with the CR fed pigs (0.0395% in the LCR group, and 0.0049% in the HCR group). Abundances of the genus Trueperella (FIG. 4H) are 0.2023%, 0.0178%, and 0.0010% in the CTL, LCR, and HCR groups, respectively.

Bacterial pathogens of major zoonotic diseases like Clostridium spp. (FIG. 4B), Streptococcus spp. (FIG. 4C), Veillonella spp. (FIG. 4D), Pseudomonas spp. (FIG. 4I), Staphylococcus spp. (FIG. 4J), and Enterococcus spp. (FIG. 4K) were analyzed. The CTL group showed the highest abundance of the genera Clostridium (5.5590%), Streptococcus (5.0596%), and Veillonella (2.8486%). These genera were 1.94-fold, 2.92-fold and 9.28-fold higher than the LCR group, and were 2.19-fold, 3.30-fold, and 131.2-fold higher than the HCR group, respectively. CR decreased the genera abundance of Pseudomonas, Staphylococcus, and Enterococcus compared to the CTL group. The LCR and HCR groups showed the lower abundances among these three genera than the CTL group. The CTL group showed the highest abundance of the genera Pseudomonas (0.1155%), Staphylococcus (0.1372%), and Enterococcus (0.1076%). Moreover, in CTL group, genera Pseudomonas and Staphylococcus were 58.5-fold and 34.8-fold higher than the LCR group, and Pseudomonas, Enterococcus, and Staphylococcus were 19.5-fold, 36.3-fold, and 13.9-fold higher than the HCR group, respectively.

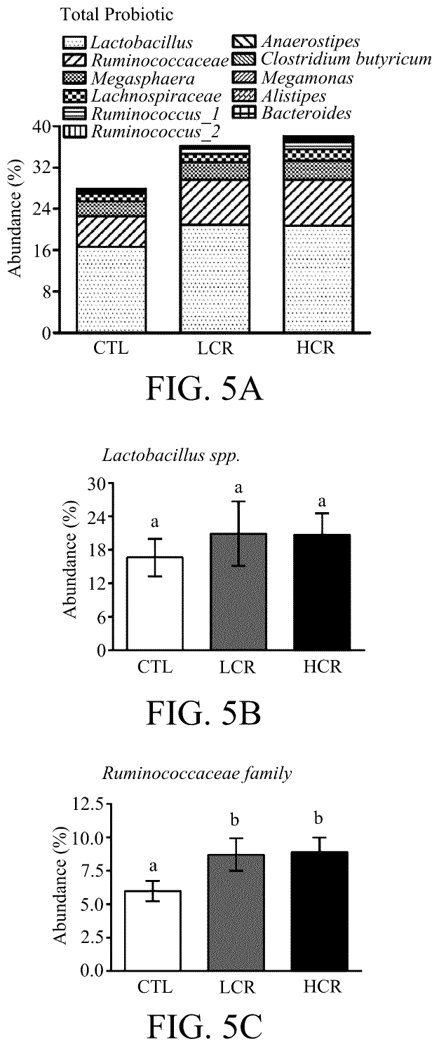

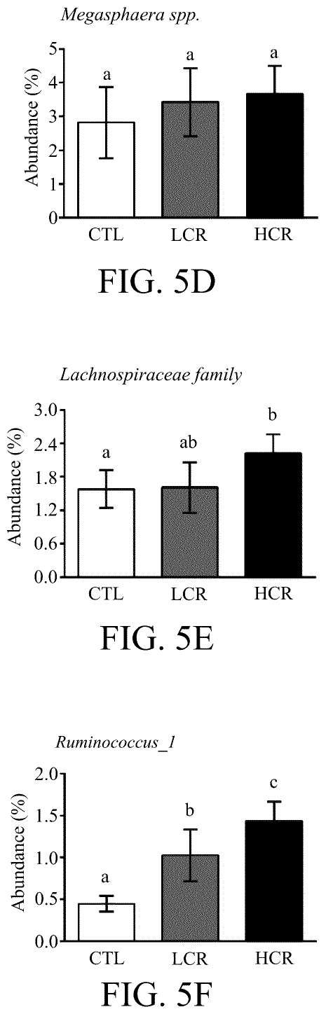

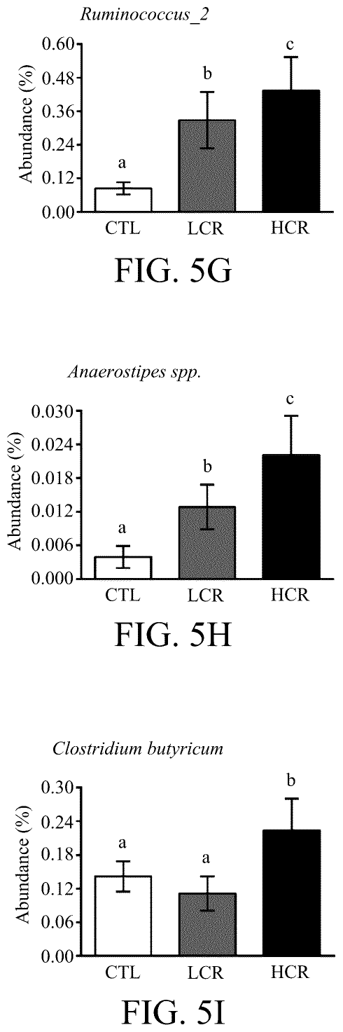

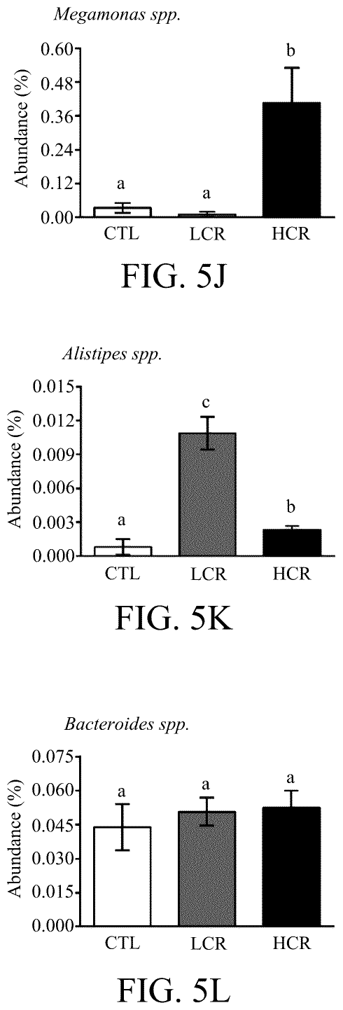

Probiotics populations in CTL, LCR and HCR animals were analyzed and shown in FIGS. 5A to 5L. The total detected probiotics in CTL, LCR, and HCR groups of animal were 27.96%, 36.61%, and 37.73%, indicating CR supplements indeed significantly increased the gut probiotic population in weaned pigs. Among the detected genera strains, Lactobacillus spp. (FIG. 5B) was the most abundant genera in the three groups with 16.63%, 20.88% and 19.07% in CTL, LCR and HCR, respectively. Seven groups of probiotic bacteria, Ruminococcaceae family (FIG. 5C), Megasphaera spp. (FIG. 5D), Lachnospiraceae family (FIG. 5E), genus Ruminococcus_1 (FIG. 5F), genus Ruminococcus_2 (FIG. 5G), and Anaerostipes spp. (FIG. 5H) were higher in the LCR and HCR groups than in the CTL group. Clostridium butyricum (FIG. 5I) and Megamonas spp. (FIG. 5J) were relatively high in the HCR group, while Alistipes spp. (FIG. 5K) was highly induced in the LCR group. There is no statistical difference observed for Bacteroides spp. in the three group of animals (FIG. 5L).

### Example 6: Effect of C. rabens on Fecal IgA and Serum TNF-α Levels in Weaned Pigs

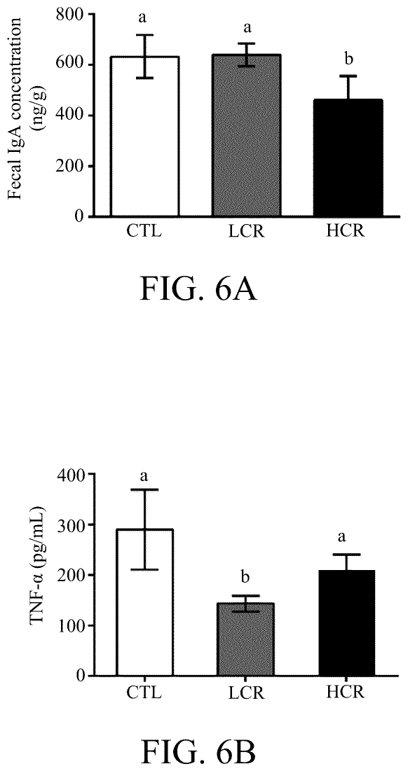

The effect of C. rabens on fecal IgA concentration of weaned pigs is showed in FIG. 6A. Fecal IgA concentration was determined at the 7th week. Concentrations of fecal IgA were 632.46 ng/g, 639.48 ng/g, and 461.21 ng/g in the CTL, LCR, and HCR groups, respectively. TNF-α, a well-known pro-inflammatory cytokine, was detected at 289.7 pg/mL in the sera of the CTL group at the 7th week, and was found to be decreased in LCR (143 pg/mL) and HCR (209.7 pg/mL) groups, as shown in FIG. 6B. These results show that CR treatment decreased the level of fecal IgA and serum TNF-α in weaned pigs.

### Example 7: Primary Metabolome Analysis of Serum from CR-Treated Weaned Pigs

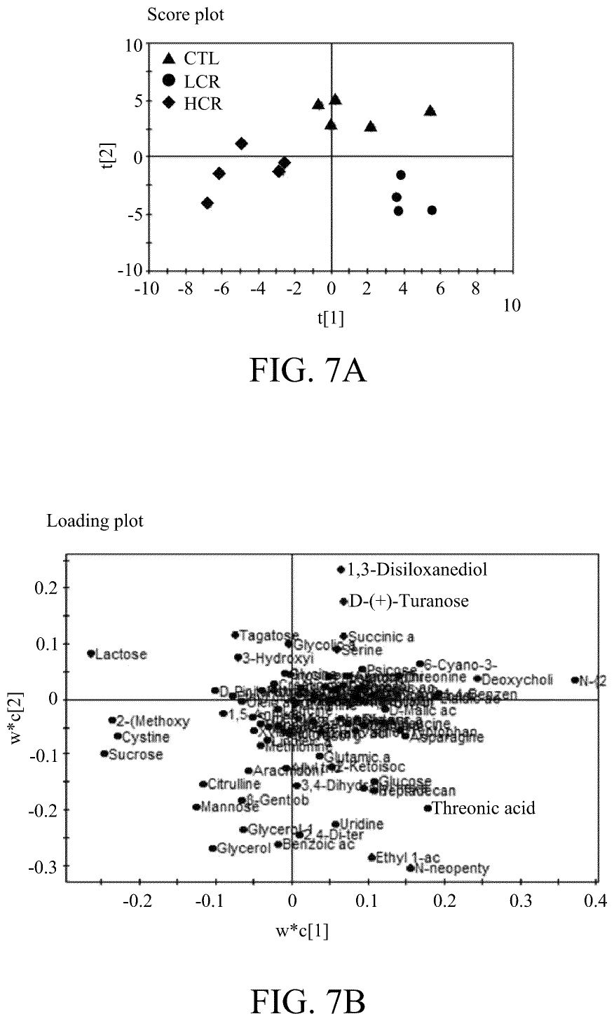

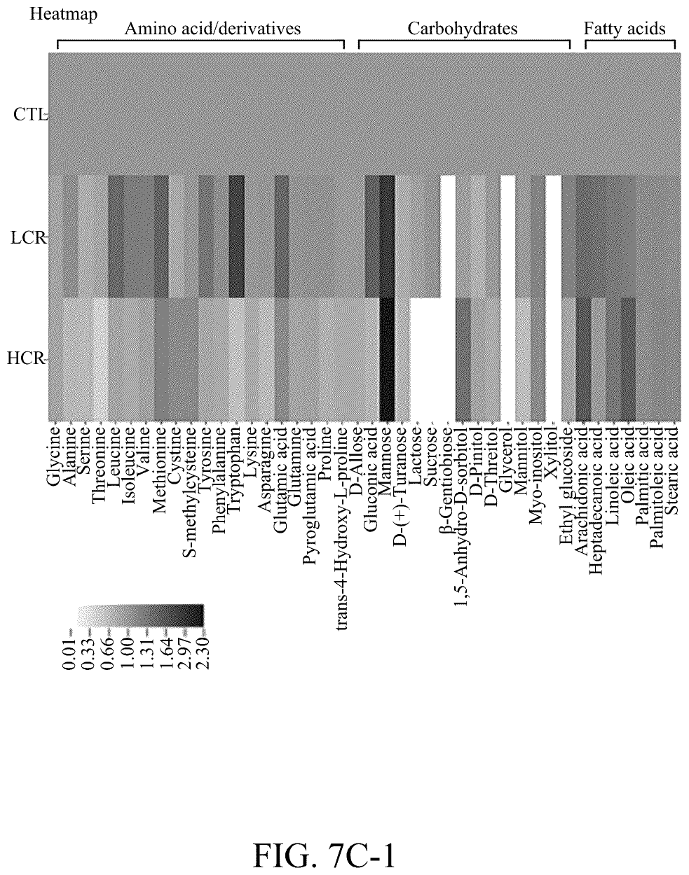

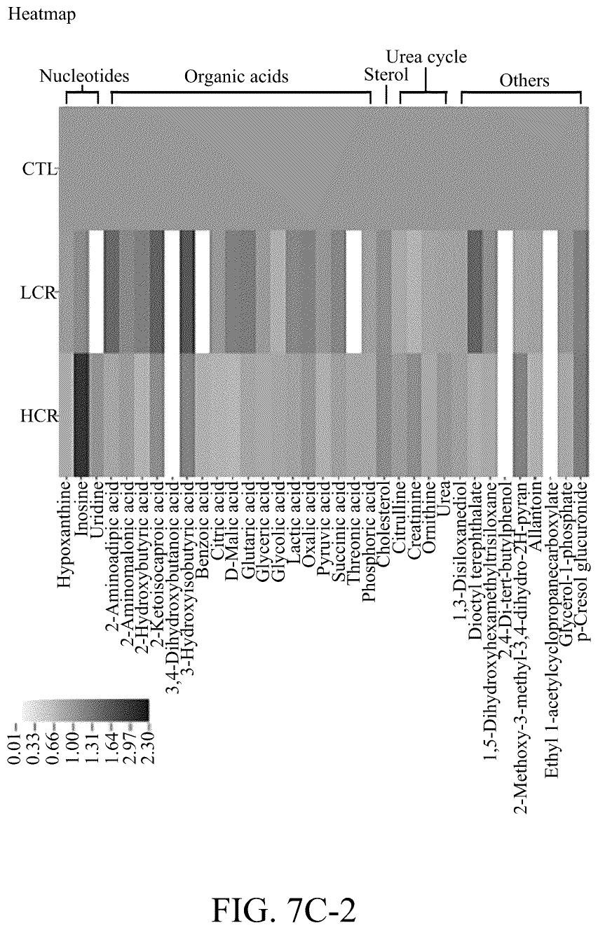

The primary metabolome is considered as one of the factors in growth performance of weaned pigs. The potential effect of CR treatment on pig metabolism was investigated using GC/Q-TOF MS. A total of 78 metabolites detected in serum were subjected to multivariate partial least squares discriminant analysis (PLS-DA). The score plot as shown in FIG. 7A revealed distinct clustering of each group, suggesting CR treatment might affect primary metabolism in weaned pigs. The corresponding loading plot shown in FIG. 7B identifies the metabolite variables which have more significant changes between the control and the CR-treated animals.

The identified metabolites were categorized and grouped according to their chemical structures and functionalities, including amino acids, carbohydrates, fatty acids, nucleotides, organic acids, sterol, urea cycle and others. FIGS. 7C-1 and 7C-2 show the heatmap of the fold change of primary metabolites from CR-fed pig serum compared to the CTL group.

Among the 20 amino acids and other metabolites identified in the LCR group, significant increase of glutamic acid (1.5-fold), leucine (1.5-fold), methionine (1.5-fold) and tryptophan (1.8-fold) were observed, while in the HCR group, threonine was decreased (0.5-fold). It is known that amino acids provide supplementary nourishment to support intestine development and growth performance of piglets. For post-weaned pigs, glutamic acid, as a dietary supplement, acts mainly in energy generation and synthesis of molecules associated with intestinal renewal. Glutamic acid has also been demonstrated to improve feed efficiency in weaned piglets (L. S. Santos, G. M. Miassi, M. L. P. Tse, L. M. Gomes, P. N. Berto, J. C. Denadai, F. R. Caldara, D. B. Dalto, and D. A. Berto, “Growth performance and intestinal replacement time of ¹³C in newly weaned piglets supplemented with nucleotides or glutamic acid.” Livestock Science 27, 160-165 (2019)).

In this embodiment, it is shown that feeding with LCR elevated the serum level of glutamic acid of weaned pigs.

In addition, leucine, a branched chain amino acid, was found significantly decreased in the HCR group. Previous studies have reported that excess dietary leucine influences metabolism of other branched-chain amino acids in growing pigs which impaired growth performance, nitrogen retention, and reduced hypothalamic serotonin synthesis (W. B. Kwon, K. J. Touchette, A. Simongiovanni, K. Syriopoulos, A. Wessels, and H. H. Stein, “Excess dietary leucine in diets for growing pigs reduces growth performance, biological value of protein, protein retention, and serotonin synthesis.” Journal of Animal Science 97, 4282-4292 (2019)). The lower leucine level detected in weaned pigs fed with HCR implies better growth performance of the tested animals.

Moreover, several studies have investigated the metabolism of threonine because of the production of Thr-rich immune system metabolites, such as immunoglobulins, and intestinal mucins, during immune system stimulation (M. O. Wellington, J. K. Htoo, A. G. Van Kessel, and D. A. Columbus, “Impact of dietary fiber and immune system stimulation on threonine requirement for protein deposition in growing pigs.” Journal of Animal Science 96, 5222-5232 (2018)). In this embodiment, it is shown that the threonine expression level in serum from pigs fed with HCR was reduced which indicates a lower degree of immune stimulation.

On the other hand, the sulfur-containing amino acid methionine is involved in pig growth and is a limiting amino acid in dietary ingredient mixtures for growing pigs (N. Litvak, A. Rakhshandeh, J. K. Htoo, and C. F. M. de Lange, “Immune system stimulation increases the optimal dietary methionine to methionine plus cysteine ratio in growing pigs.” Journal of Animal Science 91, 4188-4196 (2013)). Methionine has been reported to be involved in the pig immune system because it enhances the proliferation of immune-related cells during immune challenge. Methionine serves as a methyl donor for DNA methylation and polyamine synthesis. CR administered as a feed additive in the present disclosure resulted in higher levels of methionine detected in piglet serum and indicates that CR helps to maintain a healthy immune system in piglets.

Tryptophan is an immediate precursor of the monoaminergic neurotransmitter serotonin and has been demonstrated to decrease stress hormones, reduce aggressive behaviors, and improve growth performance in weaned pigs. CR used as feed supplement enhances the tryptophan level in piglet serum and helps to provide the beneficial effects of tryptophan.

For the effects of CR on carbohydrates metabolites, fifteen carbohydrates were identified, and 7 of them were changed (increase >1.5-fold or decrease <0.5-fold), which counted for 47% of the identified carbohydrates. In the LCR group, gluconic acid and mannose were upregulated 1.5- and 1.9-fold, respectively, while β-gentiobiose, glycerol, and xylitol were lower than the CTL group. The high dose CR diet increased the level of mannose (2.3-fold) but lowered the levels of lactose, sucrose, 0-gentiobiose, glycerol and xylitol compared with the CTL group.

Carbohydrates serve as the main source of energy in living organisms. Complex carbohydrates, after consumed by animal, are digested into simple monosaccharides for metabolism and absorption, including glucose, fructose, and galactose. In the present embodiment, increased levels of monosaccharides (gluconic acid, mannose, psicose, and tagatose) and decreased levels of disaccharides (lactose, sucrose, and β-gentiobiose) were found in the serum from pigs fed with CR. Mannose was significantly up-regulated in CR-fed pigs. Mannose is a hexose, which can be converted into glucose for catabolism or be derived from glucose for glycan biosynthesis. In swine, most carbohydrates are hydrolyzed into monosaccharides in the small intestine; therefore, the different sources and levels of carbohydrates influence the physiology and health of the gastrointestinal tract.

For the effects of CR on fatty acids and other metabolites, seven fatty acids were detected in the serum. Among them, arachidonic acid and oleic acid levels were greater in the LCR and HCR groups (1.5- to 1.6-fold) than the CTL group. Inosine, a purine nucleoside, was increased 1.9-fold in pig sera of the HCR group, whereas uridine was decreased in the LCR group. Among the 17 organic acids detected in the sera samples, 2-aminoadipic acid, 2-ketoisocaproic acid, and 3-hydroxyisobutyric acid levels were elevated (1.5- to 1.6-fold) in the LCR group, whereas 3,4-dihydroxybutanoic acid, benzoic acid, and threonic acid were decreased.

Arachidonic acid, which is the precursor of prostaglandin E2 (PGE2) and is regarded as one of the potent stimulators of bone formation, elevation of pig body weight and regional bone mineral density by arachidonic acid, have been validated (H. A. Weiler, “Dietary supplementation of arachidonic acid is associated with higher whole body weight and bone mineral density in growing pigs.” Pediatric Research 47, 692-697 (2000)). Oleic acid, a monounsaturated co-9 fatty acid, was also found to be significantly increased in CR-fed pig serum. A prior finding showed that feeding high oleic acid-containing peanuts to growing-finishing swine resulted in an increase of total unsaturated fatty acids, which have been proved to improve growth performance and meat quality of pigs.

Previous studies showed that nucleotides involve in immune response and microflora at the neonatal stage. Inosine, which has been shown to have immunotherapeutic activity, was raised in CR-fed pigs. With LCR supplementation, there is more consumption of sera uridine, and it has been shown to benefit intestinal development and health, thereby improving the growth performance and reducing the risk of diarrhea in early-weaned pigs.

### Example 8: Correlation Between Gut Microbiome and Serum Primary Metabolites in CR-Treated Weaned Pigs

The 16S sequencing data were annotated based on the Kyoto Encyclopedia of Genes and Genomes (KEGG) database to identify potentially associated KEGG pathways, as shown in Table 6 below. In level 1 prediction, the highest relative abundant pathway was related to metabolism. Further level 2 prediction in the category of metabolism, carbohydrate metabolism was the most abundant relative to other metabolisms. Because 47% of the identified carbohydrates by GC/Q-TOF MS analysis were changed significantly in the LCR and HCR fed animals, the functional correlation between carbohydrate-related probiotics (at the genus level) and carbohydrate metabolites was analyzed by Spearman's rank correlation coefficients (r).

TABLE 6
Functional analysis of fecal microbiomes
of weaned pig by KEGG database

| | Relative abundance (%) | | |
|---|---|---|---|
| | CTL | LCR | HCR |
| KEGG pathways level 1 | | | |
| Metabolism | 0.4645 | 0.4620 | 0.4650 |
| Environmental information processing | 0.1469 | 0.1497 | 0.1457 |
| Unclassified | 0.1362 | 0.1352 | 0.1358 |
| Cellular processes | 0.0262 | 0.0286 | 0.0288 |
| Human diseases | 0.0073 | 0.0071 | 0.0071 |
| Organismal systems | 0.0065 | 0.0062 | 0.0066 |
| None | 0.0018 | 0.0017 | 0.0018 |
| KEGG pathways level 2 - metabolism | | | |
| Carbohydrate metabolism | 0.1060 | 0.1075 | 0.1054 |
| Amino acid metabolism | 0.0930 | 0.0919 | 0.0936 |
| Energy metabolism | 0.0565 | 0.0564 | 0.0575 |
| Nucleotide metabolism | 0.0441 | 0.0435 | 0.0433 |
| Metabolism of cofactors and vitamins | 0.0416 | 0.0407 | 0.0421 |
| Lipid metabolism | 0.0261 | 0.0264 | 0.0263 |
| Unclassified metabolism | 0.0234 | 0.0231 | 0.0231 |
| Enzyme families | 0.0220 | 0.0222 | 0.0220 |
| Glycan biosynthesis and metabolism | 0.0200 | 0.0192 | 0.0201 |
| Metabolism of terpenoids and polyketides | 0.0167 | 0.0162 | 0.0165 |
| Xenobiotics biodegradation and metabolism | 0.0159 | 0.0153 | 0.0155 |
| Metabolism of other amino acids | 0.0147 | 0.0147 | 0.0146 |
| Biosynthesis of other secondary metabolites | 0.0088 | 0.0088 | 0.0089 |

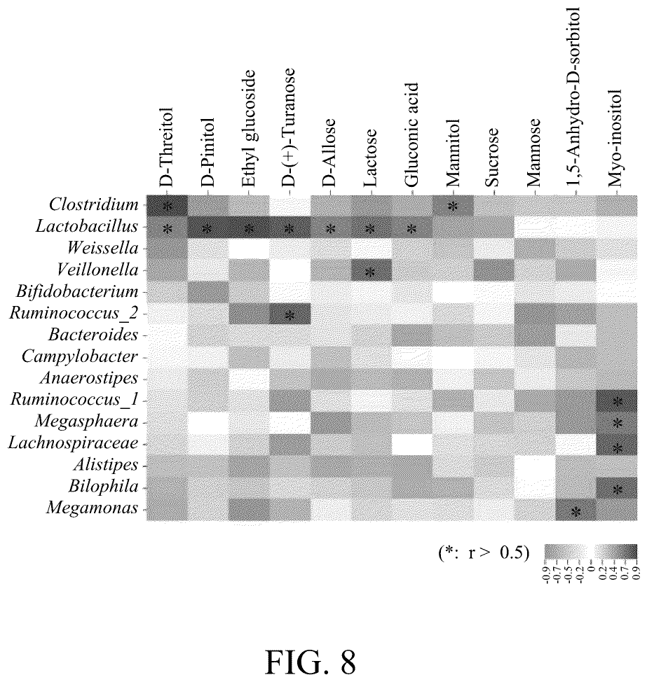

The positive correlation between probiotics and carbohydrates (r≥0.5) are shown in FIG. 8 and Table 7 below. The genus Lactobacillus was positively and significantly correlated with seven carbohydrates with the strongest positive correlation with ethyl glucoside, D-pinitol, D-(+)-turanose, lactose, D-allose, gluconic acid, and D-threitol (r=0.75-0.5). Clostridium butyricum was one of the probiotics that was positively correlated with D-threitol (r=0.77) and mannitol (r=0.5). Myo-inositol, a carbocyclic sugar serving as a participant in hormones and neurotransmitter transductions showed a significant positive correlation with the genus Ruminococcus_1 (r=0.7), genus Megasphaera (r=0.53), family Lachnospiraceae (r=0.63), and genus Bilophila (r=0.59). Lactose was positively correlated with Veillonella spp. (r=0.59). These data reveal that the populations of various probiotics were significantly enhanced in gut microbiota of weaned piglets feeding with CR that have positive impact on the primary metabolism in the fed animals on carbohydrate metabolisms.

TABLE 7
Spearman's rank correlation coefficients (r ≥ 0.5)
and relationship between probiotics and carbohydrates

| Probiotics | Carbohydrates | r |
|---|---|---|
| Lactobacillus | Ethyl glucoside | 0.75 |
| | D-Pinitol | 0.72 |
| | D-(+)-Turanose | 0.67 |
| | Lactose | 0.57 |
| | D-Allose | 0.50 |
| | Gluconic acid | 0.50 |
| Clostridium butyricum | D-Threitol | 0.50 |
| | D-Threitol | 0.77 |
| | Mannitol | 0.50 |
| Ruminococcus_1 | Myo-inositol | 0.70 |
| Ruminococcus_2 | D-(+)-Turanose | 0.64 |
| Megasphaera | Myo-inositol | 0.53 |
| Lachnospiraceae | Myo-inositol | 0.63 |
| Bilophila | Myo-inositol | 0.59 |
| Veillonella | Lactose | 0.59 |
| Megamonas | 1,5-Anhydro-D-sorbitol | 0.54 |

### Example 9: Effect of CR Extracts (CRE) and CRE Mixed with Mint Essential Oil (CREV) on Growth Performance in Weaned Pigs

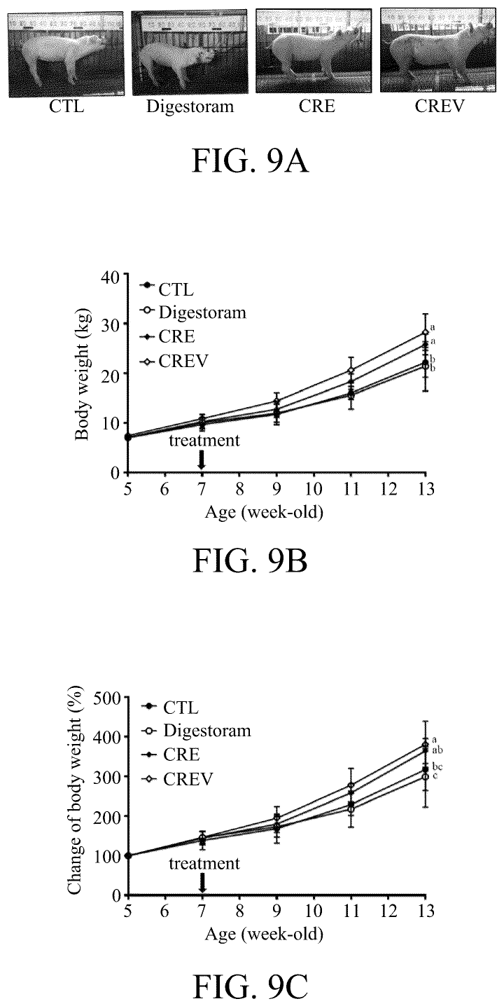

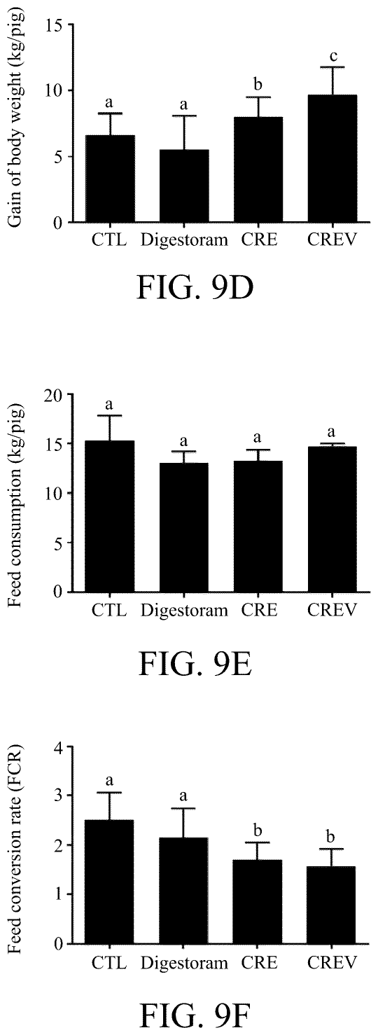

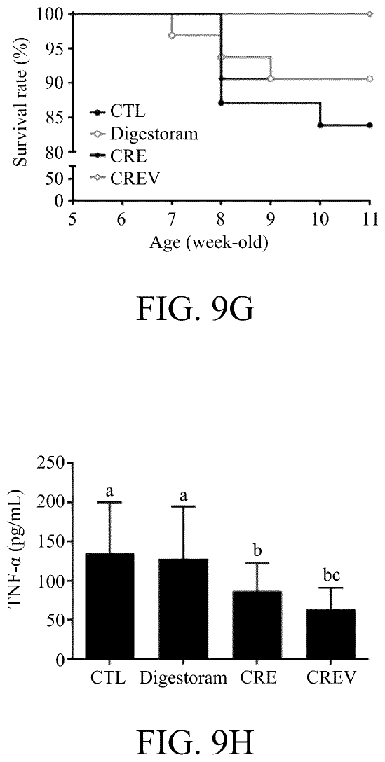

The weaned piglets were fed with basal diet or basal diet supplemented with Digestoram, CRE, and CREV, respectively for four weeks, starting from 7-week-old to 11-week-old. The growth performance in the four groups of weaned pigs is summarized in FIGS. 9A to 9H. The body shape/size and physical condition of the tested weaned piglets are shown in FIG. 9A. The CRE and CREV fed animals were stronger and bigger in size than those of animals in CTL group and Digestoram group. The mean of body weight (kg) (FIG. 9B) and change of body weight (%) (FIG. 9C) of all piglets in each group were recorded from 5-week-old to 13-week-old. The data show that after feeding with CRE or CREV containing diets for four weeks (at 11-week-old), or switching all animals back to basal diets for two weeks (at 13-week-old), the CRE and CREV groups all showed better growth performance than the CTL and Digestoram groups with statistical significance (P<0.05). At 13-week-old, the change of body weights in CRE, CREV, CTL, and Digestoram groups were revealed 363.9%, 380.9%, 316.9%, and 298.8%, respectively, relative to their respective body weights at 5-week-old (100%) (FIG. 9C).

To calculate feed conversion rate (FCR) in the four groups of weaned pig, gain of body weight per pig (kg) and feed consumption (kg/pig) after feeding for 4 weeks (at 11-week-old) were recorded. The averaged body weight gain per capital of weaned pig after feeding 28 days were 9.7 kg, 8 kg, 6.7 kg, and 5.5, respectively in CREV, CRE, CTL, and Digestoram groups (FIG. 9D). Although the feed consumption (kg/pig) among the 4 groups had no difference (FIG. 9E), the FCR was significantly decreased in CRE (1.7) and CREV (1.6) groups compared to Digestoram (2.2) group and CTL (2.5) group (FIG. 9F).

The survival rates or disease caused death of tested weaned pigs in the four groups were also recorded (FIG. 9G). At the 4 weeks feeding experiment, CREV group had the highest survival rate (100%), and the animal survival rate in CRE, Digestoram, and CTL groups were 93.8%, 90.7%, and 81.3%, respectively. Overall, CRE and CREV possess superior effect to the commercial phytogenic product Digestoram on promoting growth performance, decreasing FCR, and improving survival rate or reducing disease caused death on weaned pigs.

### Serum TNF-α Levels in Tested Animals

The sera collected from the four groups, i.e., CREV, CRE, CTL, and Digestoram, were also examined for the expression level of TNF-α. As shown in FIG. 9H, After treatment for four weeks, the concentrations of proinflammatory cytokine TNF-α in sera were significantly reduced in CRE (98 pg/mL) and CREV (86 pg/mL) groups compared with CTL (135 pg/mL) and Digestoram (128 pg/mL) groups.

### Hematological Parameters in CTL, Digestoram, CRE, and CREV Groups of Weaned Pigs

The hematological parameters in the four groups (CTL, Digestoram, CRE, and CREV) of weaned pigs were analyzed. All the detected cell types in the whole blood are within the normal ranges of reference data of healthy animals in all groups (Table 8).

TABLE 8
Hematological parameters in CTL, Digestoram, CRE, and CREV groups of weaned pigs*

| Cell type | CTL | Digestoram | CRE | CREV | References^{1, 2} |
|---|---|---|---|---|---|
| WBCs (10³/μL) | 26.4 ± 4.7^{a} | 28.9 ± 11.9^{a} | 28.2 ± 10.6^{a} | 28.2 ± 5.1^{a} | 6.3~21.1¹ |
| RBCs (10³/μL) | 6.7 ± 0.5^{ab} | 6.9 ± 0.6^{a} | 6.8 ± 0.6^{ab} | 6.5 ± 0.6^{ab} | 4.4~8.6¹ |
| Hb (g/dL) | 10.4 ± 0.9^{a} | 10.8 ± 0.6^{a} | 10.7 ± 0.8^{a} | 10.2 ± 1.1^{a} | 9.0~16.2¹ |
| HCT (%) | 39.4 ± 3.7^{ab} | 41.4 ± 2.4^{ab} | 42.0 ± 3.4^{a} | 39.8 ± 4.2^{ab} | 33.9 ~45.9¹ |
| MCV (fL) | 59.0 ± 3.6^{c} | 60.0 ± 2.9^{bc} | 61.7 ± 2.8^{ab} | 61.1 ± 2.1^{abc} | 46.6~77.2² |
| MCH (pg) | 15.5 ± 0.9^{a} | 15.6 ± 1.0^{a} | 15.8 ± 0.7^{a} | 15.7 ± 0.5^{a} | 11.2~17.6² |
| MCHC (g/dL) | 26.3 ± 0.6^{a} | 26.0 ± 0.7^{ab} | 25.6 ± 0.70^{b} | 25.6 ± 0.6^{b} | 19.6~28.0² |
| Platelets (10³/μL) | 480.1 ± 130.4^{a} | 531.7 ± 151.3^{a} | 429.5 ± 117.0^{a} | 405.8 ± 173.9^{a} | 220~665¹ |
| Neutrophils (%) | 39.0 ± 14.2^{a} | 21.9 ± 14.0^{c} | 26.2 ± 7.0^{bc} | 35.0 ± 19.2^{ab} | — |
| Lymphocytes (%) | 55.6 ± 14.2^{a} | 75.9 ± 15.1^{c} | 64.6 ± 8.4^{abc} | 63.2 ± 18.7^{ab} | 38.1~73.1¹ |
| Monocyte (%) | 3.7 ± 3.8^{bcd} | 2.0 ± 2.6^{cd} | 7.9 ± 6.8^{a} | 1.7 ± 1.3^{cd} | 0~15.0¹ |
| Eosinophils (%) | 0.9 ± 1.0^{ab} | 0.2 ± 0.4^{abc} | 1.0 ± 1.0^{a} | 0.1 ± 0.3^{bc} | 0~7.7¹ |
| Basophils (%) | 0.7 ± 1.2^{a} | 0.1 ± 0.1^{b} | 0.4 ± 0.5^{ab} | 0.0 ± 0.0^{b} | 0~1.3¹ |

*Serum samples (n = 10) of weaned pigs were collected after 4 weeks of treatment. The data are mean ± SD. ANOVA was used in statistical analysis. Different superscript letters present statistical difference within the four groups with P < 0.05. WBCs, white blood cells; RBCs, red blood cells; Hb, hemoglobin; HCT, hematocrit; MCV, mean corpuscular volume; MCH, mean corpuscular Hb; MCHC, mean corpuscular Hb concentration; Platelets, platelets count.
¹Lee Sung Pigs of National Taiwan University, Department of Animal Science and Technology and Taiwan Agricultural Technology Research Institute.
²Breeding and supply of laboratory minipigs, Livestock Research Institute, Council of Agriculture, Executive Yuan.

All references cited and discussed in this specification are incorporated herein by reference in their entireties and to the same extent as if each reference was individually incorporated by reference.

1. A method of promoting growth in an animal in need thereof, comprising:
administering an animal feed composition comprising an effective amount of a feed supplement to the animal,
wherein the feed supplement comprises Crassocephalum rabens or an extract thereof to thereby promote growth of the animal.

2. The method of claim 1, wherein the feed supplement is administered in combination with an additional herbal ingredient.

3. The method of claim 2, wherein the additional herbal ingredient is mint essential oils.

4. The method of claim 1, wherein the promotion of the growth comprises modulating gut microbiota in the animal, decreasing occurrence of at least one of diarrhea and enteric infection in the animal, increasing body weight gain of the animal, decreasing feed conversion rate, and/or modulating primary metabolism in the animal.

5. The method of claim 4, wherein the modulation of the gut microbiota comprises decreasing an amount of pathogenic gut microbiota in the animal and/or increasing an amount of probiotic microbiota in the animal.

6. The method of claim 5, wherein the pathogenic gut microbiota is at least one selected from the group consisting of Clostridium spp., Streptococcus spp., Veillonella spp., Actinobacillus spp., Escherichia_Shigella, Trueperella spp., Pseudomonas spp., Staphylococcus spp., Enterococcus spp., Pasteurella spp., Helicobacter spp., Lawsonia spp., Mycoplasma spp., and Haemophilus spp.

7. (canceled)

8. The method of claim 5, wherein the probiotic microbiota is at least one selected from the group consisting of Lactobacillus spp., Ruminococcaceae family, Megasphaera spp., Lachnospiraceae family, Ruminococcus_1, Ruminococcus_2, Megamonas spp., Clostridium butyricum, Bacteroides spp., Anaerostipes spp., and Alistipes spp.

9. (canceled)

10. The method of claim 4, wherein the at least one of the diarrhea and the enteric infection is associated with at least one bacterial pathogen selected from the group consisting of Escherichia coli, Salmonella spp., Clostridium perfringens, Campylobacter spp., Brachyspira spp., Yersinia spp., Lawsonia spp. and Helicobacter spp.

11-13. (canceled)

14. The method of claim 4, wherein the modulation of the primary metabolism comprises increasing or decreasing at least a level of an amino acid, a level of a fatty acid, a level of a carbohydrate, and a level of an organic acid or any combination thereof.

15. The method of claim 1, wherein the animal feed composition promotes survival of the animal, and/or enhances anti-inflammatory response in the animal.

16. (canceled)

17. The method of claim 15, wherein the animal feed composition lowers at least one of a TNF-α level, a fecal IgA level, or a combination thereof.

18. The method of claim 1, wherein the animal is a weaned pig or a grown-up pig.

19. (canceled)

20. The method of claim 1, wherein the Crassocephalum rabens or the extract thereof is in a powder form.

21. The method of claim 1, wherein the Crassocephalum rabens or the extract thereof ranges from about 0.001 wt % to about 5 wt % of the animal feed composition.

22. A method of preventing or treating a disease caused by porcine reproductive and respiratory syndrome virus (PRRSV) in an animal in need thereof, comprising:
administering an animal feed composition comprising an effective amount of a feed supplement to the animal,
wherein the feed supplement comprises Crassocephalum rabens or an extract thereof to thereby promote growth of the animal.

23. The method of claim 22, wherein the feed supplement is administered in combination with an additional herbal ingredient.

24. The method of claim 23, wherein the herbal ingredient is mint essential oils.

25. The method of claim 22, wherein the animal feed composition promotes survival of the animal.

26. An animal feed composition comprising a feed supplement including Crassocephalum rabens, or an extract thereof.

27. The animal feed composition of claim 26, wherein the Crassocephalum rabens or the Crassocephalum rabens extract is in a powder form.

28. The animal feed composition of claim 26, further comprising an additional herbal ingredient.

29. The animal feed composition of claim 28, wherein the additional herbal ingredient is mint essential oils.

30. The animal feed composition of claim 26, wherein the Crassocephalum rabens or the extract thereof ranges from about 0.001 wt % to about 5 wt % of the animal feed composition.
